(19) United States
(12) Patent Application Publication
(10) Pub. No.: US 2026/0274827 A1
(43) Pub. Date: Sep. 17, 2026

(54) INHIBITORS OF MYC AND USES THEREOF

(71) Applicant: Northwestern University, Evanston, IL (US)

(72) Inventors: Gary E. Schiltz, Evanston, IL (US); Sarki Abba Abdulkadir, Evanston, IL (US)

(21) Appl. No.: 19/679,355

(22) Filed: May 15, 2026

Related U.S. Application Data
(63) Continuation of application No. 19/099,534, filed on Jul. 31, 2023.
(60) Provisional application No. 63/369,892, filed on Jul. 29, 2022.

(51) Int. Cl. C07D 403/14; A61K 31/40; A61K 31/4155; A61K 31/4192; A61K 31/4196; A61K 31/427; A61K 31/4439; A61K 31/454; A61K 31/4725; A61K 31/496; A61K 31/519; A61K 31/675; A61P 35/00; C07D 207/34; C07D 231/12; C07D 231/16; C07D 231/38; C07D 249/06; C07D 249/08; C07D 401/04; C07D 401/14; C07D 403/12; C07D 405/04; C07D 417/12; C07D 417/14; C07D 487/04; C07F 9/6503

(52) U.S. Cl. CPC C07D 403/14; A61K 31/40; A61K 31/4155; A61K 31/4192; A61K 31/4196; A61K 31/427; A61K 31/4439; A61K 31/454; A61K 31/4725; A61K 31/496; A61K 31/519; A61K 31/675; A61P 35/00; C07D 207/34; C07D 231/12; C07D 231/16; C07D 231/38; C07D 249/06; C07D 249/08; C07D 401/04; C07D 401/14; C07D 403/12; C07D 405/04; C07D 417/12; C07D 417/14; C07D 487/04; C07F 9/65031

(57) ABSTRACT

c-MYC inhibitors are provided. Methods of making and using the same are also provided.

### CROSS-REFERENCE TO RELATED APPLICATIONS

This application is a continuation of U.S. patent application Ser. No. 19/099,534, filed Jan. 29, 2025, which claims the benefit of priority of U.S. Provisional Patent Application No. 63/369,892, filed Jul. 29, 2022, each of which is incorporated by reference herein in its entirety.

### STATEMENT REGARDING FEDERALLY SPONSORED RESEARCH OR DEVELOPMENT

This invention was made with government support under grant numbers CA180995 and CA257258 awarded by the National Institutes of Health. The government has certain rights in the invention.

### BACKGROUND

The c-MYC proto-oncogene is a transcription factor involved in cell cycle progression, cell growth, and metabolism. When it becomes dysregulated, the c-MYC oncogene plays a causal role in many human cancers. MYC is the most common oncogene involved in human cancers and is overexpressed in up to half of all cancers. Further, previous studies have determined that c-MYC inhibition profoundly affects tumor growth and survival in multiple models. Therefore, developing c-MYC inhibitors is among the most attractive potential anti-cancer strategies. Unfortunately, due to the difficulty in targeting transcription factors with small molecules, c-MYC is currently regarded as “undruggable.” Compounds selectively targeting c-MYC-driven cell proliferation and interfering with binding of c-MYC to DNA can be found in, e.g., U.S. Publication No. 2017/0253581, published on Sep. 7, 2017, U.S. Publication No. 2019/0062281, published on Feb. 28, 2019, U.S. Publication No. 2020/0390894, published on Dec. 17, 2020, and U.S. Publication No. 2020/0392116, published on Dec. 17, 2020, each of which is incorporated by reference herein in its entirety.

A need remains for novel compounds that interfere with binding of c-MYC to DNA, causing tumor growth inhibition (TGI), which may be used for the treatment of cell proliferation diseases and disorders such as cancer.

### SUMMARY

In one aspect, a compound is provided, the compound having Formula I:

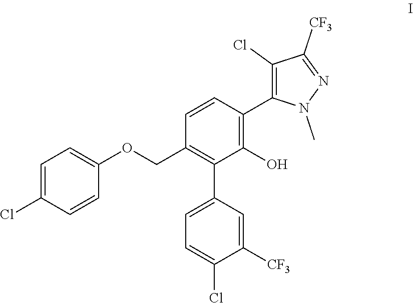 [SMILES: Cn1nc(C(F)(F)F)c(Cl)c1-c1ccc(COc2ccc(Cl)cc2)c(-c2ccc(Cl)c(C(F)(F)F)c2)c1O]

The compound having Formula I is referred to in U.S. patent application Ser. No. 19/099,534 as MYCi 606, NUCC-0226606, ET42365-228-P1, and A02B01C07D01_P1B. In this application, where possible, the compound having Formula I will be referred to as MYCi 606.

In another aspect, a pharmaceutical composition is provided, the pharmaceutical composition comprising MYCi 606 and a suitable pharmaceutical carrier, excipient, or diluent.

In another aspect, a method for treating cancer is provided, the method comprising administering the compound comprising MYCi 606 or the pharmaceutical composition comprising MYCi 606 to a subject having the cancer. In one aspect, the cancer is selected from multiple myeloma, leukemia, non-small cell lung cancer, colon cancer, cancer of the central nervous system, melanoma, ovarian cancer, renal cancer, prostate cancer, and breast cancer.

### BRIEF DESCRIPTION OF THE FIGURES

FIG. 1 shows pharmacokinetic (PK) data (concentration in plasma (ng/mL) over time) for MYCi 605 relative to MYCi 975 after oral administration (“p.o.”) (1 mg/kg) to female C57BL/6 mice.

FIG. 2 shows PK data (concentration in plasma (ng/mL) over time) for MYCi 606 relative to MYCi 975 after oral administration (1 mg/kg) to female C57BL/6 mice.

FIG. 3 shows PK data (concentration in plasma (ng/mL) over time) for MYCi 7037 relative to MYCi 975 after oral administration (1 mg/kg) to female C57BL/6 mice.

FIG. 4 depicts the tumor volume change in tested mice after oral administration of 100 mg/kg of MYCi 975 to the mice once per day.

FIG. 5 depicts the tumor volume change in tested mice after oral administration of 100 mg/kg of MYCi 605 to the mice once per day.

FIG. 6 depicts the tumor volume change in tested mice after oral administration of 30 mg/kg and 10 mg/kg of MYCi 605 to the mice once per day.

FIG. 7 depicts the tumor volume change in tested mice after oral administration of 100 mg/kg, 30 mg/kg, and 10 mg/kg of MYCi 606 to the mice once per day.

FIG. 8 depicts the tumor volume change in tested mice after oral administration of 100 mg/kg, 30 mg/kg, and 10 mg/kg of MYCi 7037 to the mice once per day.

FIG. 9 depicts the body weight change in tested mice after oral administration of 100 mg/kg of MYCi 975 to the mice once per day.

FIG. 10 depicts the body weight change in tested mice after oral administration of 100 mg/kg of MYCi 605 to the mice once per day.

FIG. 11 depicts the body weight change in tested mice after oral administration of 30 mg/kg and 10 mg/kg of MYCi 605 to the mice once per day.

FIG. 12 depicts the body weight change in tested mice after oral administration of 100 mg/kg, 30 mg/kg, and 10 mg/kg of MYCi 606 to the mice once per day.

FIG. 13 depicts the body weight change in tested mice after oral administration of 100 mg/kg, 30 mg/kg, and 10 mg/kg of MYCi 7037 to the mice once per day.

### DETAILED DESCRIPTION

### Definitions

Unless otherwise specified or indicated by context, the terms “a,” “an,” and “the” mean “one or more.” For example, “a compound” should be interpreted to mean “one or more compounds.” As used herein, “about,” “approximately,” “substantially,” and “significantly” will be understood by persons of ordinary skill in the art and will vary to some extent on the context in which they are used. If there are uses of these terms that are not clear to persons of ordinary skill in the art given the context in which they are used, “about” and “approximately” will mean plus or minus ≤10% of the particular term, and “substantially” and “significantly” will mean plus or minus >10% of the particular term. As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term that permits additional elements, but only if those additional elements do not materially affect the basic and novel characteristics of the invention claimed. As used herein, the term “subject” may be interchangeable with “patient” or “individual” and means an animal, which may be a human or non-human animal, in need of treatment. A “subject in need of treatment” may include a subject having a disease, disorder, or condition that is responsive to therapy with a compound or pharmaceutical composition as described herein, which is targeted to c-MYC for degradation of c-MYC. For example, a “subject in need of treatment” may include a subject having a cell proliferative disease, disorder, or condition such as cancer (e.g., cancers such as multiple myeloma, leukemia, non-small cell lung cancer, colon cancer, cancer of the central nervous system, melanoma, ovarian cancer, renal cancer, prostate cancer, and breast cancer). As used herein, the phrase “effective amount” shall mean that drug dosage that provides the specific pharmacological response for which the drug is administered in a significant number of subjects in need of such treatment. An effective amount of a drug that is administered to a particular subject in a particular instance will not always be effective in treating the conditions/diseases described herein, even though such dosage is deemed to be a therapeutically effective amount by those of skill in the art. As used herein, “IC50” or Half Maximal Inhibitory Concentration, is a measure of a compound's potency, indicating the concentration needed to inhibit a specific biological process or component (such as cell growth) by 50% in laboratory settings. A lower IC50 value means a compound is more potent because less of it is needed to achieve 50% inhibition, suggesting lower potential toxicity.

MYCi 606 should be interpreted as encompassing salts, solvates, and polymorphs thereof. MYCi 606 may exhibit one or more biological activities. MYCi 606 may inhibit binding of the Myc/Max complex to DNA (e.g., in a DNA gel shifting assay). In some aspects, MYCi 606 inhibits binding of the Myc/Max complex to DNA by at least 50%, 60%, 70%, 80%, 90%, 95%, 96%, 97%, 98%, or 99% at a concentration of less than about 100 μM, 50 μM, 10 μM, 1 μM, 0.1 μM, 0.05 μM, 0.01 μM, 0.005 μM, or 0.001 μM, or less. MYCi 606 may not produce significant DNA damage (e.g., in an rH2AX staining assay at a concentration greater than about 0.001 μM, 0.005 μM, 0.01 μM, 0.1 μM, 1.0 μM, 10 μM, 100 μM, or higher). MYCi 606 may inhibit the growth of cells that express c-Myc (preferably by at least 50%, 60%, 70%, 80%, 90%, 95%, 96%, 97%, 98%, or 99% at a concentration of less than about 100 μM, 50 μM, 10 μM, 1 μM, 0.1 μM, 0.05 μM, 0.01 μM, 0.005 μM, 0.001 μM, or less). MYCi 606 may not inhibit the growth of cells that do not express c-Myc (preferably by not more than 50%, 40%, 30%, 20%, 10%, 5%, 4%, 3%, 2% or less at a concentration of greater than about 0.001 μM, 0.005 μM, 0.01 μM, 0.5 μM, 0.1 μM, 1.0 μM, 10 μM, and 100 μM or higher). Concentration ranges also are contemplated herein, for example, a concentration range bounded by end-point concentrations selected from 0.001 μM, 0.005 μM, 0.01 μM, 0.5 μM, 0.1 μM, 1.0 μM, 10 μM, and 100 μM. MYCi 606 may be effective in inhibiting cell proliferation of cancer cells, including cancer cells that express c-MYC and whose proliferation is inhibited by inhibiting the biological activity of c-MYC. MYCi 606 may be effective in inhibiting cell proliferation of one or more types of cancer cells including: multiple myeloma cells, such as MM.1S cells; leukemia cells, such as CCRF-CEM, HL-60(TB), MOLT-4, RPMI-8226 and SR; non-small lung cancer cells, such as A549/ATCC, EKVX, HOP-62, HOP-92, NCI-H226, NCI-H23, NCI-H322M, NCI-H460 and NCI-H522; colon cancer cells, such as COLO 205, HCC-2998, HCT-116, HCT-15, HT29, KM12 and SW-620; CNS: SF-268, SF-295, SF-539, SNB-19, SNB-75 and U251; melanoma cancer cells, such as LOX IMVL, MALME-3M, M14, MDA-MB-435, SK-MEL-2, SK-MEL-28, SK-MEL-5, UACC-257 and UACC-62; ovarian cancer cells, such as IGR-OV1, OVCAR-3, OVCAR-4, OVCAR-5, OVCAR-8, NCI/ADR-RES and SK-OV-3; renal cancer cells, such as 786-0, A498, ACHN, CAKI-1, RXF 393, SN12C, TK-10 and UO-31; prostate cancer cells, such as DU-145 and PC-3; and breast cancer cells, such as MCF7, MDA-MB-231/ATCC, MDA-MB-468, HS 578T, BT-549 and T-47D. Cell proliferation and inhibition thereof by MYCi 606 may be assessed by cell viability methods disclosed in the art including colorimetric assays that utilize dyes such as MTT, XTT, and MTS to assess cell viability. MYCi 606 may have an IC50 of less than about 10 μM, 5 μM, 1 μM, 0.5 μM, 0.01 μM, 0.005 μM, 0.001 μM or lower in the selected assay. MYCi 606 may be formulated as an anti-cancer therapeutic, including hematologic malignancies, breast, lung, pancreas, and prostate malignancies. MYCi 606 also may be formulated as an anti-inflammation therapeutic. MYCi 606 may be formulated as a pharmaceutical composition that includes: (a) a therapeutically effective amount of MYCi 606; and (b) one or more pharmaceutically acceptable carriers, excipients, or diluents. The pharmaceutical composition may include MYCi 606 in a range of about 0.1 to 2000 mg (e.g., about 0.5 to 500 mg or about 1 to 100 mg). MYCi 606 may be administered to provide MYCi 606 at a daily dose of about 0.1 to 100 mg/kg body weight (e.g., about 0.5 to 20 mg/kg body weight or about 0.1 to 10 mg/kg body weight). In some aspects, after the pharmaceutical composition is administered to a subject (e.g., after about 1, 2, 3, 4, 5, or 6 hours post-administration), the concentration of MYCi 606 at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM). MYCi 606 and pharmaceutical compositions comprising MYCi 606 may be administered in methods of treating a subject in need thereof. For example, in the methods of treatment a subject in need thereof may include a subject having a cell proliferative disease, disorder, or condition such as cancer (e.g., cancers such as multiple myeloma, leukemia, non-small cell lung cancer, colon cancer, cancer of the central nervous system, melanoma, ovarian cancer, renal cancer, prostate cancer, and breast cancer). In some aspects of the disclosed treatment methods, the subject may be administered a dose of MYCi 606 as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some aspects, the subject may be administered a dose of MYCi 606 as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of MYCi 606 may include doses falling within dose ranges having as end-points any of these disclosed doses (e.g., 2.5 mg-200 mg). In some aspects, a minimal dose level of MYCi 606 for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some aspects, a maximal dose level of MYCi 606 for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of MYCi 606 for achieving therapy in the disclosed methods of treatment may include dose levels falling within ranges having as end-points any of these disclosed dose levels (e.g., 500-2000 ng/kg body weight of the subject). MYCi 606 may be formulated as a pharmaceutical composition in solid dosage form, although any pharmaceutically acceptable dosage form is envisioned. Example solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. MYCi 606 may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste. MYCi 606 may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride. Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose. Suitable disintegrants include lightly crosslinked polyvinyl pyrrolidone, corn starch, potato starch, maize starch, and modified starches, croscarmellose sodium, cross-povidone, sodium starch glycolate, and mixtures thereof. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present. MYCi 606 may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules. MYCi 606 may be administered in conventional dosage forms prepared by combining the active ingredient with standard pharmaceutical carriers or diluents according to conventional procedures well known in the art. These procedures may involve mixing, granulating, and compressing or dissolving the ingredients as appropriate to the desired preparation. Pharmaceutical compositions comprising MYCi 606 may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formations may be prepared by any method known in the art of pharmacy, for example by bringing into association the active ingredient with the carrier(s) or excipient(s). Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions. Pharmaceutical compositions adapted for transdermal administration may be presented as discrete patches intended to remain in intimate contact with the epidermis of the recipient for a prolonged time. For example, the active ingredient may be delivered from the patch by iontophoresis. Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams. For applications to the eye or other external tissues, for example the mouth and skin, the pharmaceutical compositions may be applied as a topical ointment or cream. When formulated in an ointment, MYCi 606 may be employed with either a paraffinic or a water-miscible ointment base. Alternatively, MYCi 606 may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent. Pharmaceutical compositions adapted for nasal administration where the carrier is a solid include a coarse powder having a particle size (e.g., in the range 20 to 500 microns), which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient. Pharmaceutical compositions adapted for parenteral administration include aqueous and non-aqueous sterile injection solutions which may contain antioxidants, buffers, bacteriostats, and solutes, which render the formulation isotonic with the blood of the intended recipient; and aqueous and non-aqueous sterile suspensions, which may include suspending agents and thickening agents. The formations may be presented in unit-dose or multidose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Tablets and capsules for oral administration may be in unit dose presentation form and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents. MYCi 606 or pharmaceutical compositions comprising MYCi 606 may be administered in methods of treatment. For example, MYCi 606 or pharmaceutical compositions comprising MYCi 606 may be administered in methods of treating cell proliferative diseases and disorders. Cell proliferative diseases and disorders treated by the disclosed methods may include, but are not limited to, cancers selected from the group consisting of multiple myeloma, leukemia, non-small cell lung cancer, colon cancer, cancer of the central nervous system, melanoma, ovarian cancer, renal cancer, prostate cancer, and breast cancer. Optionally, MYCi 606 or pharmaceutical compositions comprising MYCi 606 may be administered with additional therapeutic agents, optionally in combination, to treat cell proliferative diseases and disorders. In some aspects, one or more additional therapeutic agents are administered with MYCi 606 or with pharmaceutical compositions comprising MYCi 606, where the additional therapeutic agent is administered prior to, concurrently with, or after administering MYCi 606 or the pharmaceutical compositions comprising MYCi 606. In some aspects, the pharmaceutical composition is formulated to comprise MYCi 606 and further to comprise one or more additional therapeutic agents, for example, one or more additional therapeutic agents for treating cell proliferative diseases and disorders. In some aspects, additional therapeutic agents may include, but are not limited to, therapeutic agents for treating leukemias and lymphomas, such as acute myeloid leukemia (AML), acute lymphocytic leukemia (ALL), chronic myelogenous leukemia (CML), and non-Hodgkin's lymphoma. In some aspects, additional therapeutic agents may include, but are not limited to, antimetabolite antineoplastic agents that inhibit the synthesis of DNA. Suitable antimetabolite antineoplastic agents that inhibit the synthesis of DNA may include, but are not limited to, nucleoside and/or nucleotide derivatives. Suitable nucleoside and/or nucleotide derivatives may include, but are not limited to, cytosine arabinoside (ara-C), otherwise called cytarabine.

### EXAMPLES

The following Examples are illustrative and are not intended to limit the scope of the claimed subject matter.

### Example 1: Synthesis of MYCi 606 from Core A6

“Core A6” was synthesized according to the scheme below and extracted and analyzed using liquid chromatography mass spectroscopy (LC/MS).

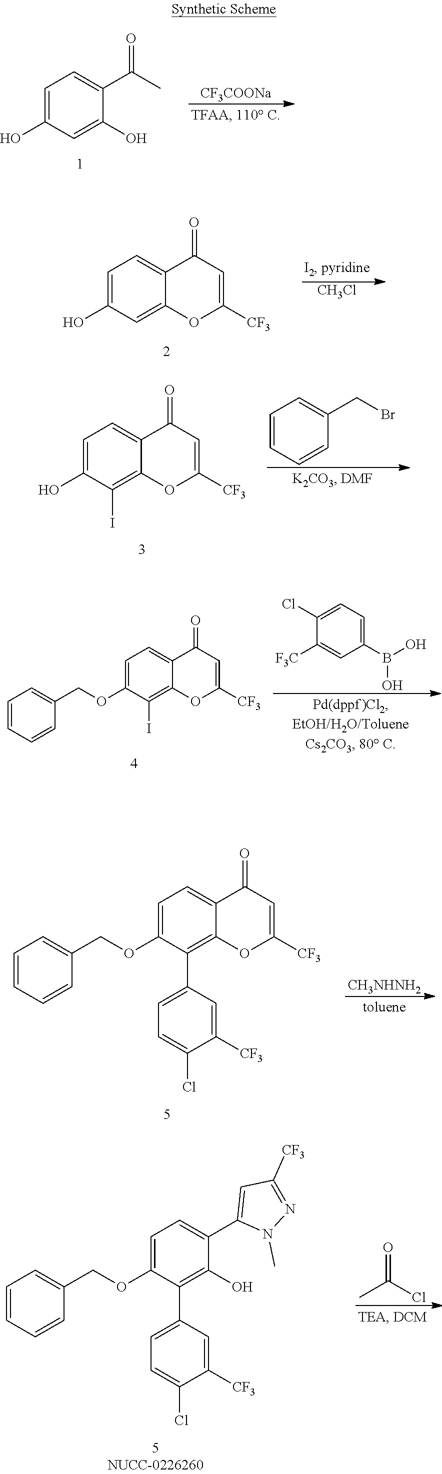

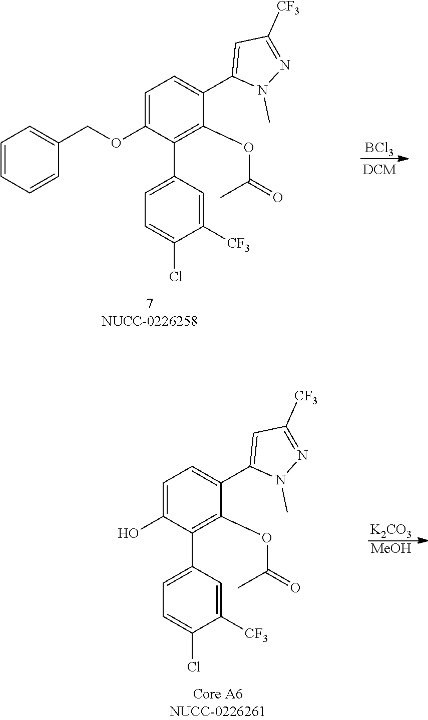

Experimental details were as follows:

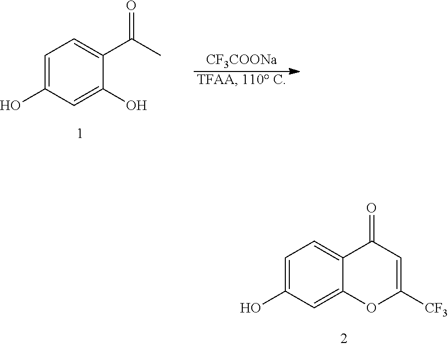

General procedure for compound 2. A suspension of compound 1 (10 g, 65.73 mmol, 8.47 mL, 1 eq) in TFAA (37 mL) was placed in a high-pressure tube (100 mL). Sodium trifluoroacetate (19.67 g, 144.60 mmol, 2.2 eq) was added, and the system was capped and stirred at 130° C. for 24 h. LCMS showed that all starting materials were consumed. The reaction was allowed to cool to 25° C. The batch was diluted with EtOAc. The mixture was neutralized by saturated aqueous K₂CO₃ solution until no bubbling was observed. The organic layer was separated, and the aqueous portion was extracted with EtOAc (3×500 mL). The organic layer was washed with brine, dried over anhydrous Na₂SO₄, and concentrated to ⅓ volume of EtOAc. The flask was allowed to stand at 25° C. for 8 hours. Compound 2 (180 g, 793.0 mmol, 40.0% yield) was collected as a white solid.

### Reaction LCMS:

### LCMS Method:

LCMS (ESI+): m/z=231.1; (M+H)+, RT: 0.806 min.

5_95AB_2 min-220-254-ELSD: LCMS. The gradient was 5% B in 0.40 min and 5-95% B at 0.40-3.00 min, hold on 95% B for 1.00 min, and 95-5% B in 0.01 min. The flow rate was 1.0 mL/min. Mobile phase A was 0.037% trifluoroacetic acid in water, mobile phase B was 0.018% trifluoroacetic acid in acetonitrile. The column used for chromatography was a Kinetex C18 50*2.1 mm column (5 μm particles). Detection methods were diode array (DAD) and evaporative light scattering (ELSD) detection as well as positive electrospray ionization. Mass Spectrometry (MS) range was 100-1000.

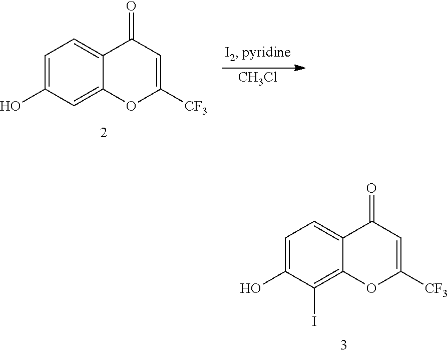 [SMILES: O=c1cc(C(F)(F)F)oc2cc(O)ccc12 ; "CH3Cl" ; O=c1cc(C(F)(F)F)oc2c(I)c(O)ccc12]

General procedure for compound 3. A solution of compound 2 (180 g, 782.13 mmol, 1 eq), iodine (794.05 g, 3.13 mol, 630.20 mL, 4 eq), and pyridine (247.47 g, 3.13 mol, 252.52 mL, 4 eq) in chloroform (1 L) was stirred at 25° C. for 8 h. LCMS showed the reaction completed. The mixture was poured into water (500 mL) and triturated with petroleum ether:ethyl acetate (10:1, 800 mL) to give compound 3 (210 g, 589.83 mmol, 75.41% yield) as a yellowish solid.

### Reaction LCMS:

### LCMS Method:

LCMS (ESI+): m/z=356.9 (M+H)+, RT: 0.890 min. 5_95AB_2 min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000. 1H NMR (400 MHz, METHANOL-d4) δ ppm 6.79 (s, 1H) 7.02 (d, J=8.82 Hz, 1H) 7.95-7.99 (m, 1H).

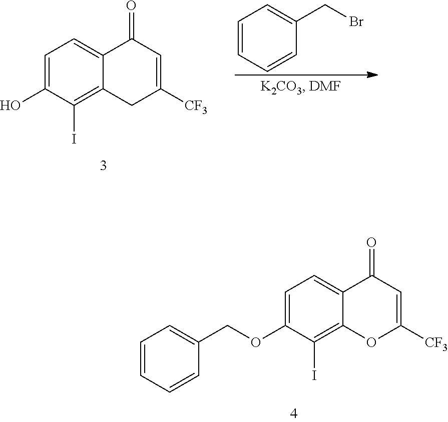 [SMILES: O=C1C=C(C(F)(F)F)Cc2c1ccc(O)c2I ; BrCc1ccccc1 ; O=c1cc(C(F)(F)F)oc2c(I)c(OCc3ccccc3)ccc12]

General procedure for compound 4. To a solution of compound 3 (130 g, 365.13 mmol, 1 eq), bromomethylbenzene (74.94 g, 438.16 mmol, 52.04 mL, 1.2 eq), and DMF in acetone (1 L) was added potassium carbonate (100.93 g, 730.26 mmol, 2 eq). The mixture was stirred at 80° C. for 8 h. TLC showed the reaction completed. The mixture was poured into water (500 mL) and extracted with ethyl acetate (3×500 mL). The organic layer was dried over Na₂SO₄ and concentrated to give crude product, which was purified by chromatography on silica and eluted with petroleum ether:ethyl acetate=10:1 to 5:1 to give compound 4 (120 g, 268.96 mmol, 73.66% yield) as a yellowish solid. 1H NMR (400 MHz, CHLOROFORM-d) δ ppm 5.27 (s, 2H) 6.63 (s, 1H) 6.96 (d, J=8.93 Hz, 1H) 7.10-7.39 (m, 8H) 7.40-7.46 (m, 2H) 8.08 (d, J=8.93 Hz, 1H).

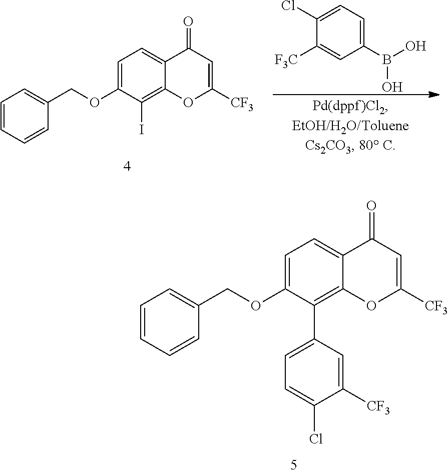 [SMILES: O=c1cc(C(F)(F)F)oc2c(I)c(OCc3ccccc3)ccc12 ; OB(O)c1ccc(Cl)c(C(F)(F)F)c1 ; O=c1cc(C(F)(F)F)oc2c(-c3ccc(Cl)c(C(F)(F)F)c3)c(OCc3ccccc3)ccc12]

General procedure for compound 5. To a solution of compound 4 (100 g, 22.41 mmol, 1 eq), [4-chloro-3-(trifluoromethyl)phenyl]boronic acid (5.03 g, 22.41 mmol, 1 eq), and cesium carbonate (14.61 g, 44.83 mmol, 2 eq) in toluene (2 L), ethanol (400 mL), and water (80 mL) was added Pd(dppf)Cl₂ (1.64 g, 2.24 mmol, 0.1 eq) under N₂ atmosphere. The mixture was stirred at 80° C. for 8 h. TLC showed the reaction completed. The mixture was poured into water (500 mL) and extracted with ethyl acetate (3×500 mL). The organic layer was dried over Na₂SO₄ and concentrated to give crude product, which was triturated with ethyl acetate:petroleum ether (1:10, 500 mL) to give compound 5 (93 g, 18.64 mmol, 83.18% yield) as a yellowish solid. 1H NMR (400 MHz, CHLOROFORM-d) δ ppm 5.26 (s, 2H) 6.71 (s, 1H) 7.27 (d, J=2.93 Hz, 2H) 7.34-7.41 (m, 3H) 7.55-7.66 (m, 2H) 7.85 (s, 1H) 8.26 (d, J=8.93 Hz, 1H).

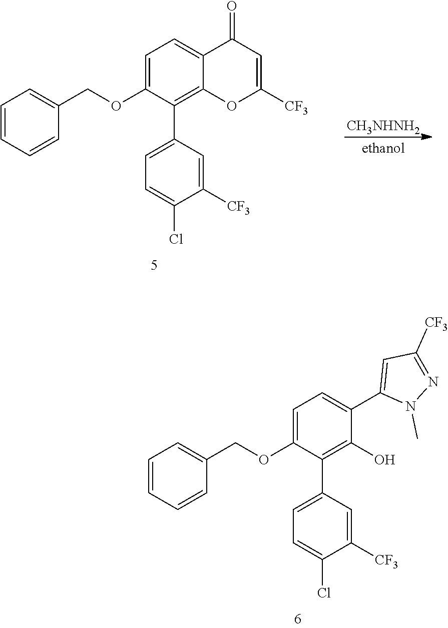

General procedure for compound 6. A suspension of compound 5 (100 g, 200.48 mmol, 1 eq) and methylhydrazine (27.71 g, 601.44 mmol, 31.67 mL, 3 eq) in ethanol (500 mL) was bubbled with N₂ gas for 10 minutes. The vial was heated at 80° C. for 16 h. LCMS showed all starting materials consumed. The resulting mixture was concentrated to give crude residue, which was triturated by toluene to obtain compound 6 (75 g, 142.35 mmol, 71.01% yield) as a brown solid. LCMS (ESI+): m/z=527.2 (M+H)+, RT: 1.203 min. 5_95AB_2 min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

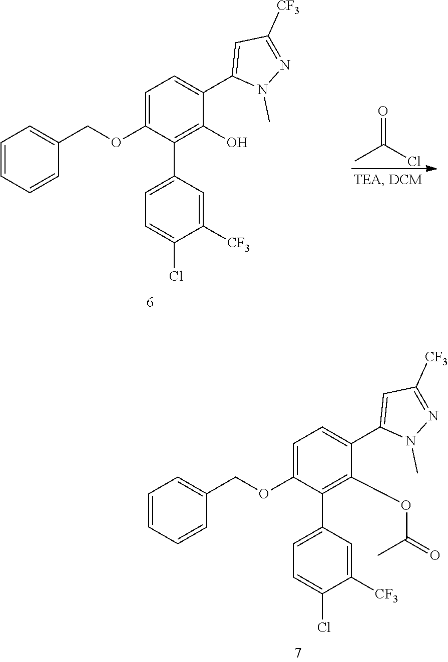

General procedure for compound 7. To a solution of compound 6 (42 g, 79.72 mmol, 1 eq) in DCM (500 mL) was added acetyl chloride (7.51 g, 95.66 mmol, 6.83 mL, 1.2 eq) and TEA (9.68 g, 95.66 mmol, 13.31 mL, 1.2 eq). The mixture was stirred at 25° C. for 8 hrs. TLC showed all starting materials consumed. Once completion, the mixture was poured into water and extracted with DCM (2×100 mL). The organic layer was dried over Na₂SO₄, concentrated to give compound 7 (45 g, 79.10 mmol, 99.23% yield) as a white solid, which was used directly in the next step. 1H NMR (400 MHz, CHLOROFORM-d) δ 1.72-1.81 (m, 3H), 3.75-3.84 (m, 3H), 5.10-5.16 (m, 2H), 6.45-6.52 (m, 1H), 7.07 (d, J=8.60 Hz, 1H), 7.21-7.27 (m, 3H), 7.38 (s, 2H), 7.44-7.49 (m, 1H), 7.51-7.57 (m, 1H), 7.72-7.76 (m, 1H).

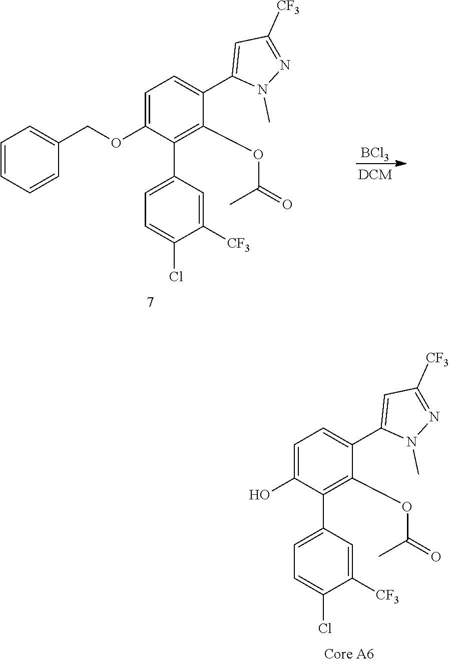 [SMILES: CC(=O)Oc1c(-c2cc(C(F)(F)F)nn2C)ccc(OCc2ccccc2)c1-c1ccc(Cl)c(C(F)(F)F)c1 ; "BCl3" ; CC(=O)Oc1c(-c2cc(C(F)(F)F)nn2C)ccc(O)c1-c1ccc(Cl)c(C(F)(F)F)c1]

General procedure for Core A6. A solution of compound 7 (50 g, 87.89 mmol, 1 eq) in DCM (200 mL) was cooled to 0° C. BCl₃ (1 M, 175.78 mL, 2 eq) was added to the mixture dropwise, and the temperature was maintained below 0° C. The mixture was stirred at 0° C. for 4 hrs. TLC showed all starting materials consumed. On completion, ice (100 mL) was added, and the organic layer was separated. The aqueous was extracted with EtOAc (3×100 mL). The organic layer was dried over Mg₂SO₄, concentrated, purified by chromatography on silica, and eluted with PE:EA=10:1 to 1:1 give Core A6 (25 g, 52.22 mmol, 59.41% yield) as a white solid. 1H NMR (400 MHz, CHLOROFORM-d) δ 1.78 (s, 3H), 3.76-3.84 (m, 3H), 5.95 (s, 1H), 6.48-6.53 (m, 1H), 6.96-7.02 (m, 1H), 7.23-7.28 (m, 1H), 7.48-7.53 (m, 1H), 7.62 (d, J=8.16 Hz, 1H), 7.70-7.74 (m, 1H).

MYCi 606 was synthesized from Core A6 according to the below scheme and extracted and analyzed using LC/MS.

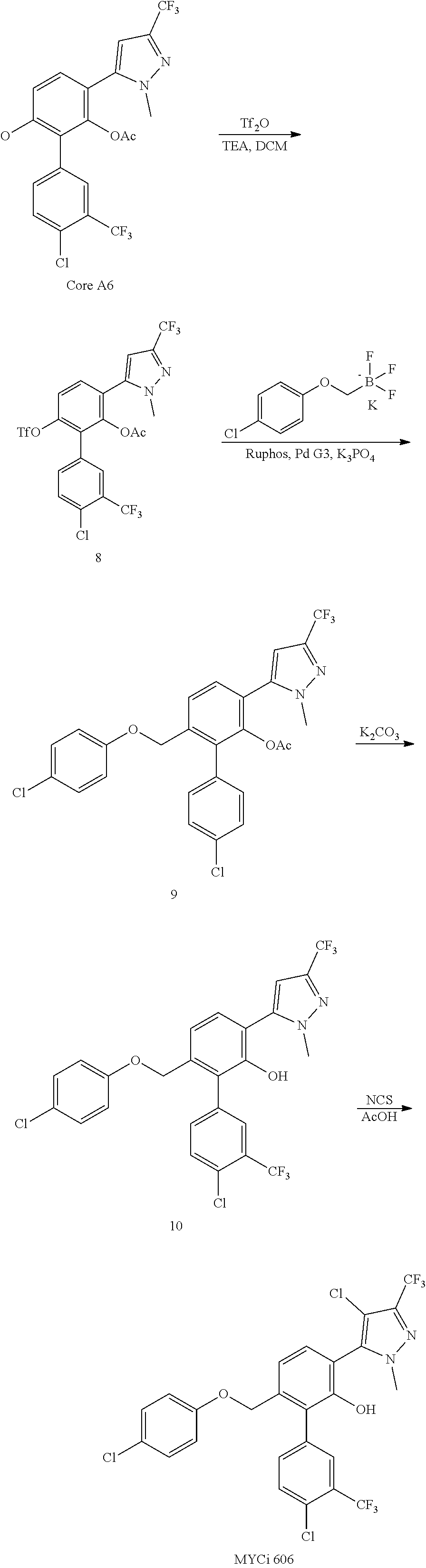

Experimental details were as follows:

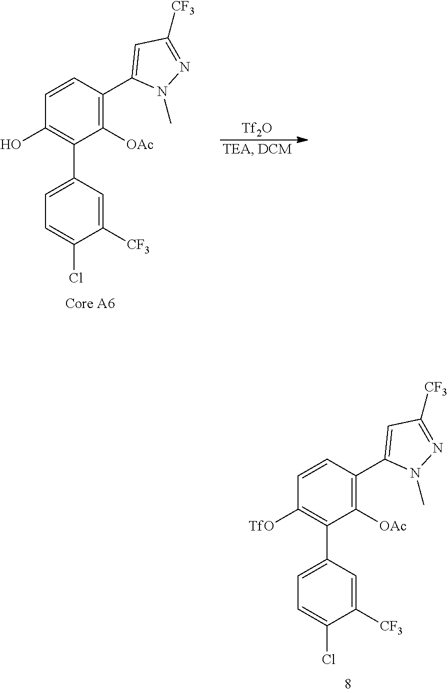 [SMILES: "Tf2O" ; CC(=O)Oc1c(-c2cc(C(F)(F)F)nn2C)ccc(O)c1-c1ccc(Cl)c(C(F)(F)F)c1 ; "TEA, DCM" ; CC(=O)Oc1c(-c2cc(C(F)(F)F)nn2C)ccc(OS(=O)(=O)C(F)(F)F)c1-c1ccc(Cl)c(C(F)(F)F)c1]

General procedure for compound 8. To a solution of Core A6 (400 mg, 835.47 μmol, 1 eq) and pyridine (264.34 mg, 3.34 mmol, 269.74 μL, 4 eq) in DCM (12 mL) was added Tf₂O (353.58 mg, 1.25 mmol, 206.77 μL, 1.5 eq) dropwise at 0° C., and the reaction was stirred at 20° C. for 3 hrs. LCMS showed the starting material was consumed, and a new peak with desired product MS was detected. The reaction was diluted with water (50 mL) and extracted with DCM (3×25 mL). The organic layer was separated, and the combined organic layer was washed with brine (30 mL), dried over Na₂SO₄, filtered, and concentrated under reduced pressure to give crude product. The crude product was purified by column chromatography on silica gel (eluted with petroleum ether/ethyl acetate=50/1 to 10/1) to give compound 8 (0.4 g, yield 78.38%) as a white solid. LCMS (ESI+): RT=0.915 min, m/z 611.1 (M+H)+. 5-95AB_2 min: LC/MS (The column used for chromatography was an Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

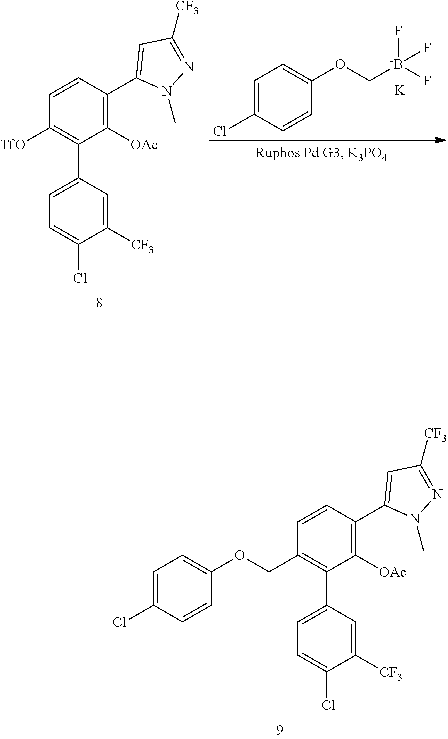

General procedure for compound 9. To a solution of compound 8 (200 mg, 327.42 μmol, 1 eq), the boronated compound shown in the scheme (188.32 mg, 757.88 μmol, 2.31 eq), and K₃PO₄ (208.50 mg, 982.26 μmol, 3 eq) in H₂O (2 mL) and toluene (10 mL) was added RuPhos Pd G3 (54.77 mg, 65.48 μmol, 0.2 eq) under nitrogen. The reaction was stirred at 110° C. for 20 hrs. LCMS showed the starting material was consumed, and a new peak with desired product MS was detected. The reaction was filtered, and the filtrate was concentrated to give the crude product. The crude product was purified by Prep-TLC (petroleum ether/ethyl acetate=2/1) to give compound 9 (50 mg, yield 20.25%) as a yellow solid. LCMS (ESI+): RT=0.988 min, m/z 603.2 (M+H)+. 5-95AB_2 min: LC/MS (The column used for chromatography was an Agilent Poroshell SB-C18 3.0*30 mm, 2.7 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B (0.70-1.16 min), 95-5% B (1.16-1.50 min). The flow rate was 1.5 mL/min.

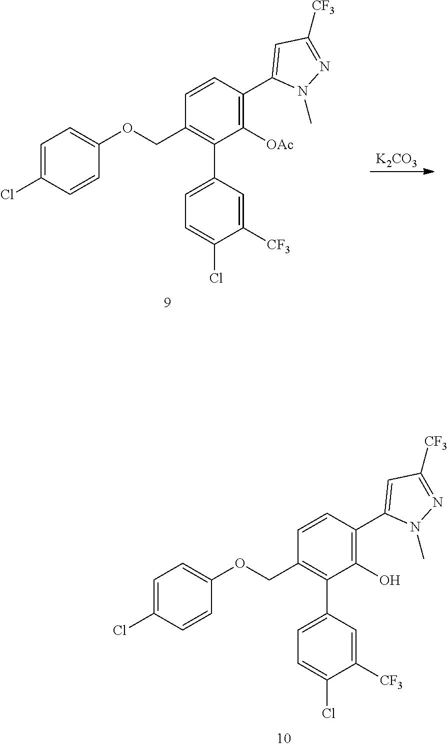

General procedure for compound 10. To a solution of Compound 9 (80 mg, 106.08 μmol, 80% purity, 1 eq) in MeOH (3 mL) was added K₂CO₃ (29.32 mg, 212.15 μmol, 2 eq). The reaction was stirred at 20° C. for 12 hrs. LCMS showed the starting material was consumed, and a new peak with desired product MS was detected. The reaction was diluted with water (10 mL) and was extracted with ethyl acetate (3×10 mL). The combined organic layers were washed with brine (10 mL) and dried over Na₂SO₄ and concentrated to give crude product. The crude product was purified by Prep-HPLC (column: Waters Xbridge Prep OBD C18150*40 mm*10 um; mobile phase: [water (10 mM NH4HCO3)-ACN]; B %: 70%-95%, 8 min) to give compound 10 (40 mg, yield 67.18%) as a white solid. LCMS (ESI+): RT=3.108 min, m/z 561.0 (M+H)+. 5-95CD_4.5 min: LC/MS (The column used for chromatography was an Xbridge C18 2.1*50 mm, 5 um). Detection methods are diode array (DAD). MS mode was positive electrospray ionization. MS range was 100-1000. Mobile phase A was 10 mM ammonium bicarbonate in water, and mobile phase B was HPLC grade acetonitrile. The gradient was 5-95% B in 4.30 min 0.5% B in 0.01 min, 5-95% B (0.01-3.00 min), and hold at 95% B within 0.5 min, 95-5% B (3.50-3.51 min), with a hold at 5% B for 0.79 min. The flow rate was 1.0 mL/min (0.01-4.30 min).

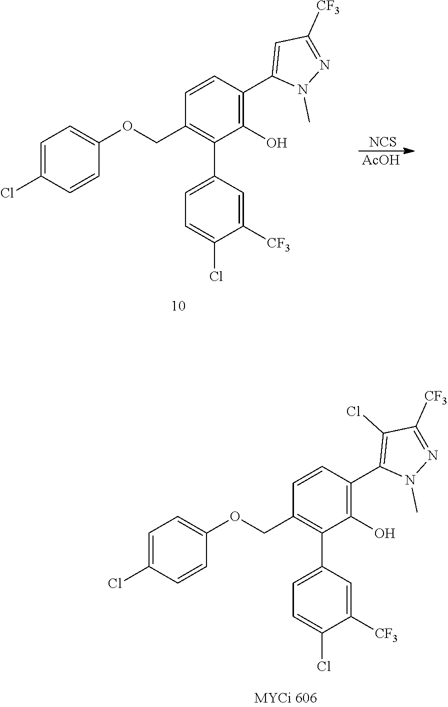 [SMILES: "AcOH" ; Cn1nc(C(F)(F)F)cc1-c1ccc(COc2ccc(Cl)cc2)c(-c2ccc(Cl)c(C(F)(F)F)c2)c1O ; "NCS" ; Cn1nc(C(F)(F)F)c(Cl)c1-c1ccc(COc2ccc(Cl)cc2)c(-c2ccc(Cl)c(C(F)(F)F)c2)c1O]

General procedure for MYCi 606. To a solution of compound 10 (50 mg, 89.08 μmol, 1 eq) in AcOH (2 mL) was added NCS (11.89 mg, 89.08 μmol, 1 eq) at 0° C. The reaction was stirred at 60° C. for 12 hrs. LCMS showed about 33% of the starting material was remaining, and two new peaks with desired Ms were detected. The reaction was concentrated to give crude product. The crude product was purified by Prep-HPLC (column: Waters Xbridge Prep OBD C18 150*40 mm*10 um; mobile phase: [water (10 mM NH4HCO3)-ACN]; B %: 70%-90%, 8 min) to give the crude product, which was further purified by Prep-HPLC (column: Phenomenex Luna 80*30 mm*3 um; mobile phase: [water (0.1% TFA)-ACN]; B %: 70%-90%, 8 min) to give MYCi 606 (15.2 mg, yield 27.9%) and a byproduct as white solid. MYCi 606: 1H NMR (ET42365-228-P1B, 400 MHz, CHLOROFORM-d) δ 3.84 (s, 3H), 4.65-4.78 (m, 2H), 4.93 (br s, 1H), 6.75 (d, J=8.88 Hz, 2H), 7.23 (d, J=8.88 Hz, 2H), 7.33-7.43 (m, 2H), 7.54 (br d, J=8.25 Hz, 1H), 7.67 (d, J=8.13 Hz, 1H), 7.76 (d, J=1.38 Hz, 1H) LCMS (ESI+): RT=2.467 min, m/z 596.9 (M+H)+. 5_95CD_6 min-220-254-ELSD: LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H2O+10 mM NH4HCO3, mobile phase B was acetonitrile. The column used for chromatography was an Xbridge C18 2.1*50 mm column (5 um particles). Detection methods are diode array (DAD) detection. MS mode was positive electrospray ionization. MS range was 100-1000. LCMS Methods:

### Example 2: Synthesis of MYCi 606 from Compound 11

MYCi 606 may be alternatively synthesized according to the scheme below.

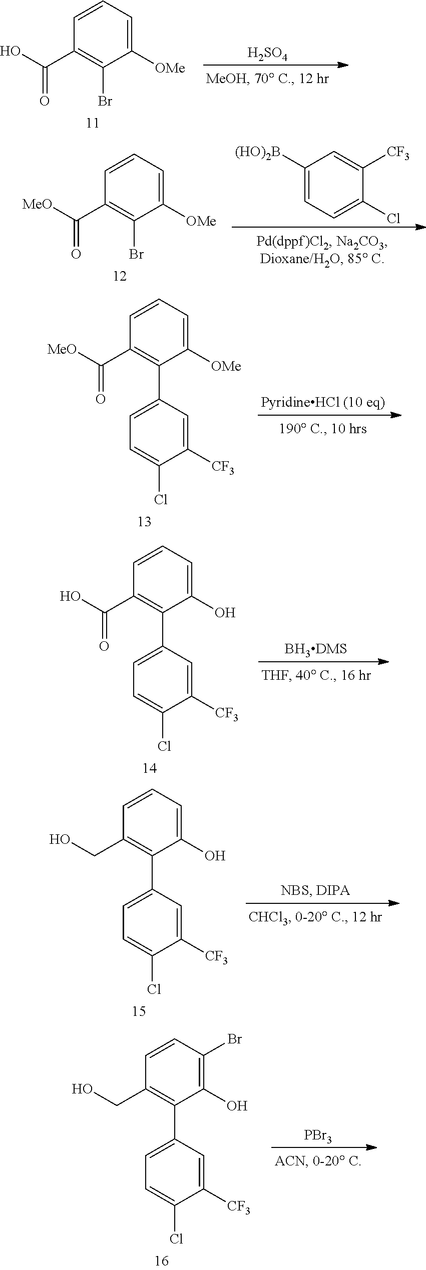

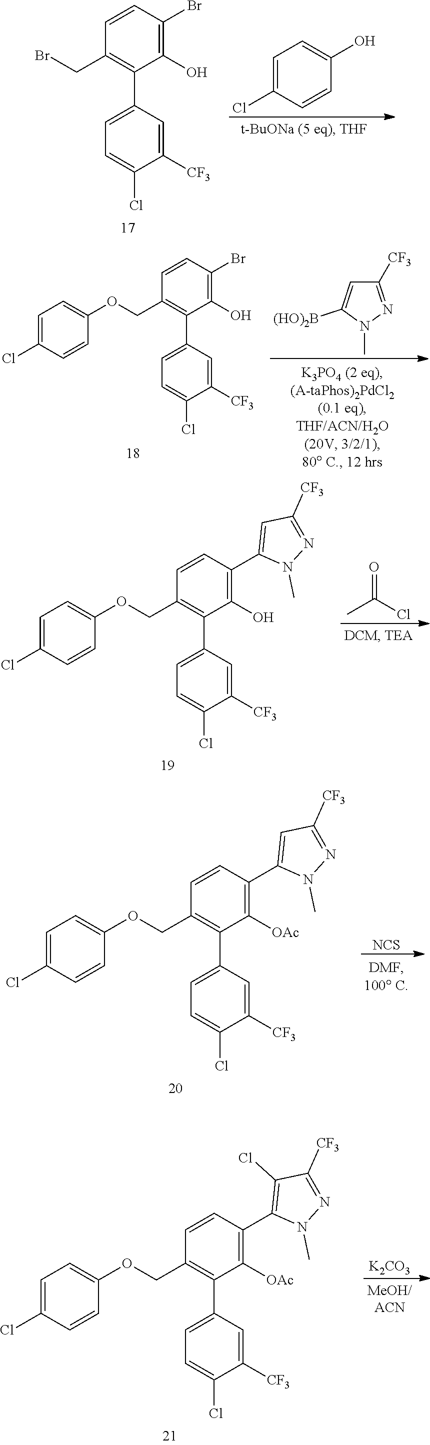

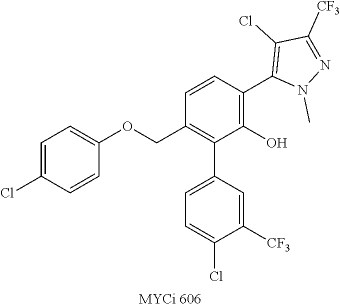 [SMILES: Cn1nc(C(F)(F)F)c(Cl)c1-c1ccc(COc2ccc(Cl)cc2)c(-c2ccc(Cl)c(C(F)(F)F)c2)c1O]

Experimental details were as follows:

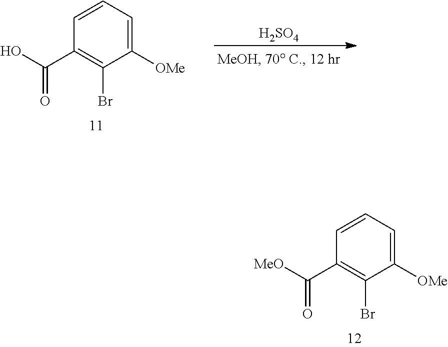 [SMILES: COc1cccc(C(=O)O)c1Br ; "H2SO4" ; COC(=O)c1cccc(OC)c1Br]

General procedure for compound 12. To a solution of compound 11 (25 g, 108.20 mmol) in MeOH (250 mL) was added sulfuric acid (9.20 g, 93.80 mmol, 5 mL) dropwise at 20° C. The mixture was heated to 70° C. and stirred for 12 hrs. TLC (petroleum ether/ethyl acetate=3/1, Rf=0.25) showed the starting material was consumed, and a new spot was generated. The reaction mixture was concentrated to give the crude product, which was diluted with water (500 mL), adjusted to pH=6~7 with K₂CO₃, and extracted with ethyl acetate (3×200 mL). The combined organic layers were washed with brine (100 mL), dried over Na₂SO₄, and concentrated to give compound 12 (76 g, 90.76% yield) as a white solid, which was used directly for next step without further purification. 1H NMR (400 MHz, CHLOROFORM-d) δ 3.93 (s, 3H), 3.94 (s, 3H), 7.02 (dd, J=8.00, 1.38 Hz, 1H), 7.26-7.28 (m, 1H), 7.30-7.36 (m, 1H).

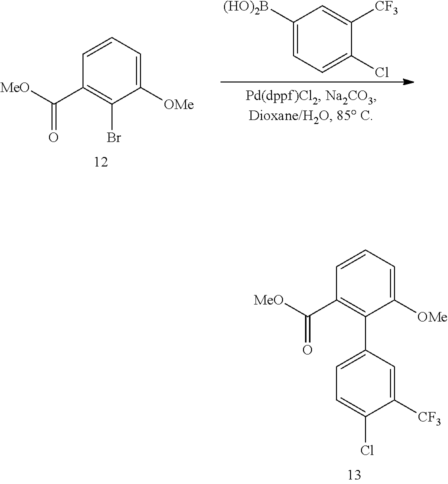 [SMILES: COC(=O)c1cccc(OC)c1Br ; OB(O)c1ccc(Cl)c(C(F)(F)F)c1 ; COC(=O)c1cccc(OC)c1-c1ccc(Cl)c(C(F)(F)F)c1]

General procedure for compound 13. To a solution of methyl 2-bromo-3-methoxy-benzoate (25 g, 102.01 mmol), [4-chloro-3-(trifluoromethyl)phenyl]boronic acid (27.47 g, 122.41 mmol) in dioxane (200 mL), and H₂O (100 mL) were added Na₂CO₃ (21.62 g, 204.02 mmol) and Pd(dppf)Cl₂·CH₂Cl₂ (4.17 g, 5.10 mmol) at 20° C. under nitrogen. The mixture was heated to 85° C. and stirred for 12 hrs. TLC (petroleum ether/ethyl acetate=10/1) showed the starting material (Rf=0.4) was consumed and new spot (Rf=0.45) generated. The reaction mixture was poured into water (800 mL) and extracted with ethyl acetate (200 mL×3). The organic layers were combined, washed with brine (2×20 mL), dried over Na₂SO₄, filtered, and concentrated under reduced pressure to give a residue. The residue was purified by column chromatography on silica gel (eluting with petroleum ether/ethyl acetate=0/1 to 20/1) to give compound 13 (85 g, 72.52% yield) as a white solid. 1H NMR (400 MHz, CHLOROFORM-d) δ 3.62 (s, 3H), 3.77 (s, 3H), 7.14 (d, J=8.00 Hz, 1H), 7.36 (dd, J=8.19, 1.44 Hz, 1H), 7.41-7.47 (m, 1H), 7.48-7.54 (m, 2H), 7.57 (d, J=1.50 Hz, 1H).

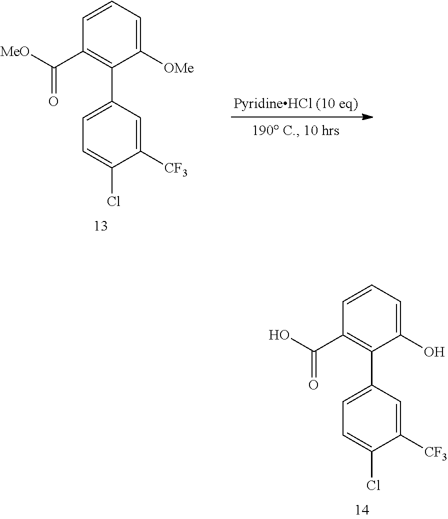 [SMILES: COC(=O)c1cccc(OC)c1-c1ccc(Cl)c(C(F)(F)F)c1 ; O=C(O)c1cccc(O)c1-c1ccc(Cl)c(C(F)(F)F)c1]

General procedure for compound 14. A mixture of methyl 2-[4-chloro-3-(trifluoromethyl)phenyl]-3-methoxy-benzoate (35 g, 101.53 mmol) and pyridine; hydrochloride (117.33 g, 1.02 mol) was heated to 190° C. and stirred for 10 hrs under nitrogen. TLC (petroleum ether/ethyl acetate=5/1) showed the starting material (Rf=0.6) was consumed, and a new spot (Rf=0.2) generated. The reaction mixture was diluted with water (1000 mL) and extracted with ethyl acetate (300 mL×3). The combined organic layers were washed with brine (100 mL×3), dried over Na₂SO₄, and concentrated to give compound 14 (90 g, 88.64% yield) as a white solid, which was used directly for next step without further purification. 1H NMR (400 MHz, DMSO-d6) δ 7.12 (d, J=7.75 Hz, 1H), 7.25-7.35 (m, 2H), 7.51 (br d, J=8.25 Hz, 1H), 7.60 (s, 1H), 7.70 (d, J=8.25 Hz, 1H), 9.88 (s, 1H), 12.70 (br s, 1H).

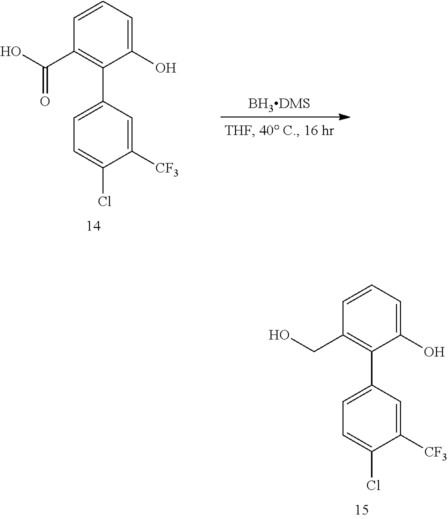 [SMILES: O=C(O)c1cccc(O)c1-c1ccc(Cl)c(C(F)(F)F)c1 ; OCc1cccc(O)c1-c1ccc(Cl)c(C(F)(F)F)c1]

General procedure for compound 15. To a solution of 2-[4-chloro-3-(trifluoromethyl)phenyl]-3-hydroxy-benzoic acid (40 g, 126.32 mmol) in THF (1000 mL) was added BH3-Me2S (10 M, 63.16 mL) dropwise at 0° C. The mixture was heated to 40° C. and stirred for 16 hrs. LCMS showed the starting material was consumed. The reaction mixture was cooled to 0° C. and slowly quenched with methanol (200 mL) at 0° C. The mixture was concentrated under reduced pressure to give a residue. The residue was purified by column chromatography on silica gel (eluting with petroleum ether/ethyl acetate=20/1 to 3/1) to give compound 15 (72 g, 89.45% yield) as a white solid. 1H NMR (400 MHz, DMSO-d6) δ 4.15 (d, J=5.25 Hz, 2H), 5.00 (t, J=5.32 Hz, 1H), 6.86 (d, J=8.00 Hz, 1H), 7.01 (d, J=7.50 Hz, 1H), 7.17-7.25 (m, 1H), 7.56 (dd, J=8.25, 1.75 Hz, 1H), 7.68 (d, J=1.75 Hz, 1H), 7.74 (d, J=8.25 Hz, 1H), 9.43 (s, 1H).

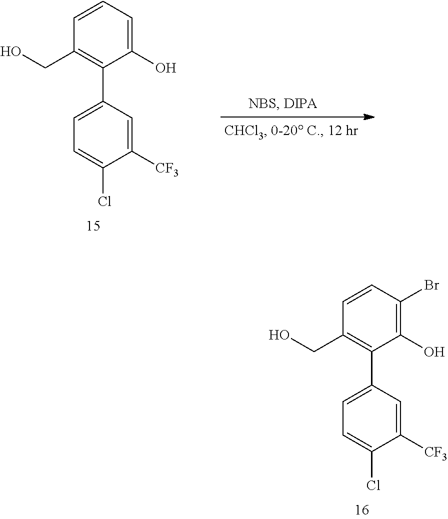 [SMILES: OCc1cccc(O)c1-c1ccc(Cl)c(C(F)(F)F)c1 ; OCc1ccc(Br)c(O)c1-c1ccc(Cl)c(C(F)(F)F)c1]

General procedure for compound 16. To a solution of 2-[4-chloro-3-(trifluoromethyl)phenyl]-3-(hydroxymethyl)phenol (30 g, 99.12 mmol) and DIPA (30.09 g, 297.35 mmol, 42.02 mL) in CHCl₃ (700 mL) was added 1-bromopyrrolidine-2,5-dione (17.64 g, 99.12 mmol) portion-wise at 0° C. The mixture was warmed up to 20° C. and stirred for 12 hrs. LCMS showed 16.1% of the starting material was remaining, and 55% product (Rt=0.854 min) with desired MS was detected. The mixture was poured into water (1000 mL) and extracted with DCM (3×300 mL). The organic layers were combined, washed with brine (2×100 mL), dried over Na₂SO₄, filtered, and concentrated under reduced pressure to give a residue. The residue was purified by column chromatography on silica gel (eluting with petroleum ether/ethyl acetate=50/1 to 10/1) to give compound 16 (40 g, 50.24% yield) as a white solid. 1H NMR (400 MHz, DMSO-d6) δ4.08 (br d, J=3.26 Hz, 2H), 5.11 (br t, J=4.64 Hz, 1H), 7.03 (d, J=8.28 Hz, 1H), 7.51-7.60 (m, 2H), 7.69 (d, J=1.88 Hz, 1H), 7.78 (d, J=8.16 Hz, 1H), 8.93 (br s, 1H).

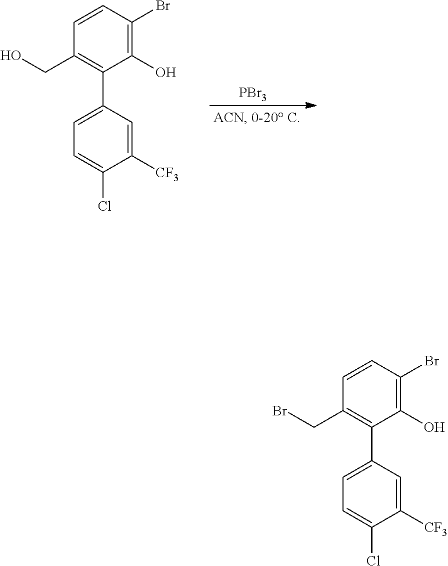 [SMILES: OCc1ccc(Br)c(O)c1-c1ccc(Cl)c(C(F)(F)F)c1 ; "PBr3" ; Oc1c(Br)ccc(CBr)c1-c1ccc(Cl)c(C(F)(F)F)c1]

General procedure for compound 17. To a solution of 6-bromo-2-[4-chloro-3-(trifluoromethyl)phenyl]-3-(hydroxymethyl)phenol (20 g, 52.41 mmol) in ACN (200 mL) was added PBr₃ (5.68 g, 20.97 mmol) dropwise at 0° C. The mixture was warmed up to 20° C. and stirred for 12 hrs. LCMS showed the starting material was consumed, and 69.7% of product (Rt=0.975 min) with desired MS was detected. The reaction mixture was poured into water (300 mL) and extracted with ethyl acetate (150 mL×3). The organic layers were combined, washed with brine (2×50 mL), dried over Na₂SO₄, filtered, and concentrated under reduced pressure to give a residue. The residue was purified by column chromatography on silica gel (eluting with petroleum ether/ethyl acetate=1/0 to 50/1) to give compound 17 (20 g, 77.26% yield) as a white solid. 1H NMR (400 MHz, DMSO-d6) δ 4.32 (br d, J=5.50 Hz, 2H), 7.07 (d, J=8.38 Hz, 1H), 7.57-7.65 (m, 2H), 7.73 (d, J=1.38 Hz, 1H), 7.84 (d, J=8.25 Hz, 1H), 9.19 (s, 1H).

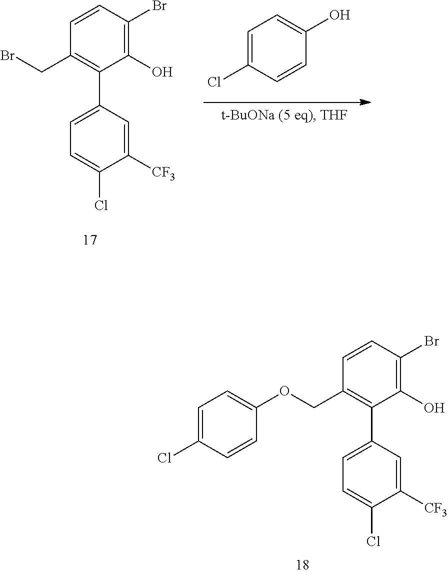 [SMILES: Oc1c(Br)ccc(CBr)c1-c1ccc(Cl)c(C(F)(F)F)c1 ; Oc1ccc(Cl)cc1 ; Oc1c(Br)ccc(COc2ccc(Cl)cc2)c1-c1ccc(Cl)c(C(F)(F)F)c1]

General procedure for compound 18. To a solution of 4-chlorophenol (28.92 g, 224.99 mmol, 22.08 mL) in a mixture solution of THF (100 mL) and DMF (100 mL) was added t-BuONa (21.62 g, 224.99 mmol). The reaction was stirred at 25° C. for 30 mins. Compound 17 (20 g, 45.00 mmol) in THF (50 mL) was added dropwise at 0° C. The reaction was stirred at 20° C. for 12 hrs. LCMS showed the starting material was consumed, and 51.9% peak (Rt=1.019 min) with desired MS was detected. TLC (petroleum ether/ethyl acetate=20/1, Rf=0.4) showed a new spot was generated. The reaction mixture was poured into water (300 mL) and extracted with ethyl acetate (3×100 mL). The organic layers were combined, washed with brine (2×50 mL), dried over Na₂SO₄, filtered, and concentrated under reduced pressure to give a residue. The residue was purified by column chromatography on silica gel (eluting with petroleum ether/ethyl acetate=1/0 to 100/1) to give compound 18 (20 g, 85.80% yield) as a white solid. 1H NMR (400 MHz, DMSO-d6) δ 4.69 (br s, 2H), 6.75-6.85 (m, 2H), 7.06 (d, J=8.25 Hz, 1H), 7.21-7.30 (m, 2H), 7.55-7.65 (m, 2H), 7.67-7.76 (m, 2H), 9.16 (s, 1H).

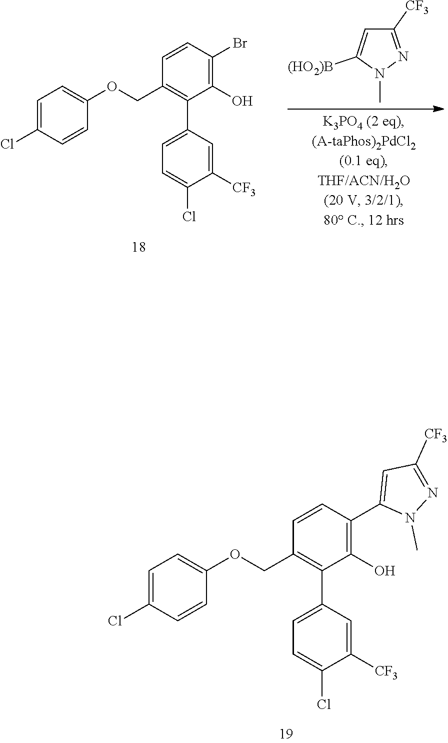

General procedure for compound 19. To a solution of compound 18 (10 g, 20.32 mmol) and [2-methyl-5-(trifluoromethyl)pyrazol-3-yl]-boronic acid (7.88 g, 40.64 mmol) in dioxane (80 mL), H2O (40 mL), and ACN (80 mL) were added 4-ditert-butylphosphanyl-N,N-dimethyl-aniline;dichloropalladium (1.44 g, 2.03 mmol) and K₃PO₄ (12.94 g, 60.96 mmol) under nitrogen at 20° C. The mixture was heated to 80° C. and stirred for 12 hrs. LCMS showed the starting material was consumed, and 73.3% peak (Rt=1.041 min) with the desired MS was detected. TLC (petroleum ether/ethyl acetate=5/1, Rf=0.5) showed that a new spot was generated. The reaction mixture was poured into water (200 mL) and extracted with ethyl acetate (60 mL×3). The organic layers were combined, washed with brine (2×50 mL), dried over Na₂SO₄, filtered, and concentrated under reduced pressure to give a residue. The residue was purified by column chromatography on silica gel (eluting with petroleum ether/ethyl acetate=1/0 to 10/1) to give Compound 9 (9 g, 71.02% yield) as a white solid. 1H NMR (400 MHz, DMSO-d6) δ 3.78 (s, 3H), 4.61-4.91 (m, 2H), 6.80 (s, 1H), 6.82-6.91 (m, 2H), 7.21-7.31 (m, 3H), 7.37 (d, J=7.88 Hz, 1H), 7.65 (dd, J=8.19, 1.69 Hz, 1H), 7.71-7.80 (m, 2H), 9.01 (s, 1H).

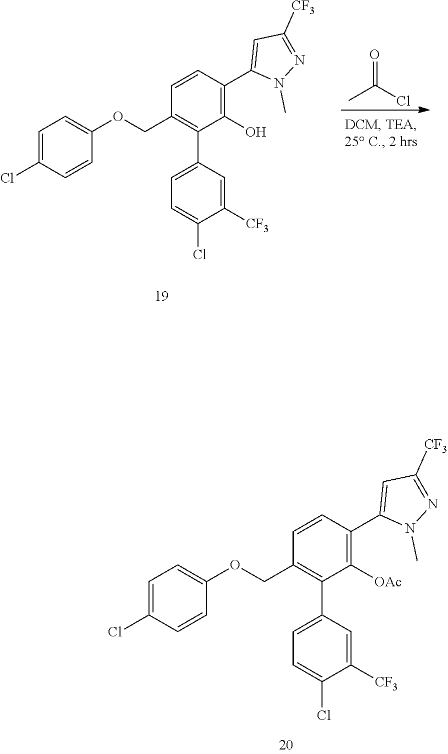

General procedure for compound 20. To a solution of compound 19 (10 g, 17.82 mmol) and TEA (2.70 g, 26.72 mmol, 3.72 mL) in DCM (150 mL) was added acetyl chloride (1.68 g, 21.38 mmol, 1.53 mL) dropwise at 0° C. The mixture was warmed up to 20° C. and stirred for 2 hrs. LCMS showed the starting material was consumed, and 93.4% peak (Rt=0.916 min) with the desired Ms was detected. TLC (petroleum ether/ethyl acetate=5/1, Rf=0.4) showed a new spot was generated. The reaction mixture was poured into water (200 mL) and extracted with DCM (60 mL×3). The organic layers were combined, washed with brine (2×20 mL), dried over Na₂SO₄, filtered, and concentrated under reduced pressure to give a residue. The residue was purified by column chromatography on silica gel (eluting with petroleum ether/ethyl acetate=50/1 to 10/1) to give compound 20 (10.2 g, 85.40% yield) as a white solid. 1H NMR (400 MHz, DMSO-d6) δ 1.71 (s, 3H), 3.82 (s, 3H), 4.80-4.99 (m, 2H), 6.78 (s, 1H), 6.84-6.91 (m, 2H), 7.24-7.32 (m, 2H), 7.61 (br d, J=7.88 Hz, 1H), 7.72 (br s, 1H), 7.75 (s, 2H), 7.79 (d, J=8.25 Hz, 1H).

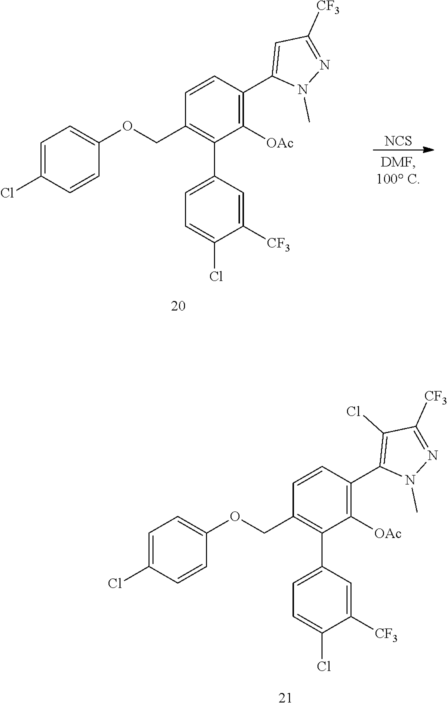 [SMILES: CC(=O)Oc1c(-c2cc(C(F)(F)F)nn2C)ccc(COc2ccc(Cl)cc2)c1-c1ccc(Cl)c(C(F)(F)F)c1 ; "NCS" ; CC(=O)Oc1c(-c2c(Cl)c(C(F)(F)F)nn2C)ccc(COc2ccc(Cl)cc2)c1-c1ccc(Cl)c(C(F)(F)F)c1]

General procedure for compound 21. To a solution of compound 20 (10 g, 16.57 mmol) in DMF (100 mL) was added 1-chloropyrrolidine-2,5-dione (8.85 g, 66.30 mmol) at 20° C. The mixture was heated to 100° C. and stirred for 12 hrs. LCMS and HPLC showed 2.5% of the starting material was remaining, and 67.3% product with desired MS was detected. The reaction mixture was poured into water (600 mL) and extracted with ethyl acetate (3×200 mL). The organic layers were combined, washed with brine (2×50 mL), dried over Na₂SO₄, filtered, and concentrated under reduced pressure to give a residue. The residue was purified by prep-HPLC to give compound 21 (4.5 g, 42.36% yield) and a byproduct, both as a white solid. Compound 21:1H NMR (400 MHz, DMSO-d6) δ 1.65 (s, 3H), 3.73 (s, 3H), 4.72-4.97 (m, 2H), 6.82 (br d, J=8.82 Hz, 2H), 7.22 (br d, J=8.82 Hz, 2H), 7.47-7.70 (m, 2H), 7.70-7.80 (m, 3H). Separation method of Prep-HPLC: Instrument: Shimadzu LC-8A preparative HPLC Column: Phenomenex luna C18 (250*70 mm, 15 um) Mobile phase: A for H2O (0.09% TFA) and B for CAN Gradient: B from 73% to 90% in 20 min Flow rate: 130 mL/min Wavelength: 220&254 nm.

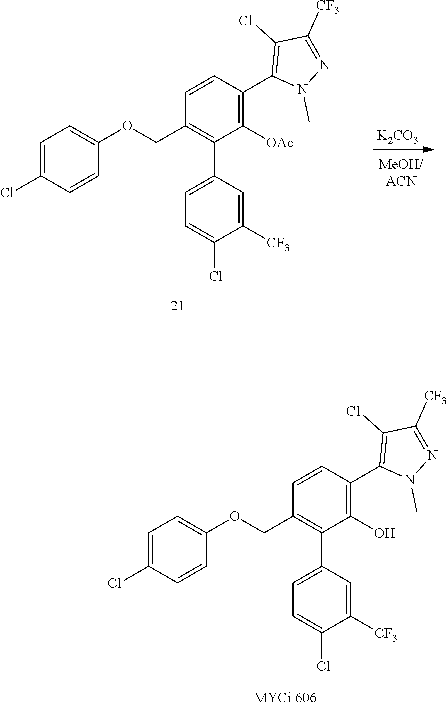

General procedure for preparation of MYCi 606. To a solution of compound 21 (6.5 g, 10.19 mmol) in MeOH (100 mL) and ACN (100 mL) was added K₂CO₃ (4.23 g, 30.57 mmol) at 20° C. The mixture was stirred for 12 hrs. LCMS showed the starting material was consumed, and 96.6% peak (Rt=0.941 min) with the desired MS was detected. The reaction mixture was concentrated under reduced pressure to remove methanol and acetonitrile. Water (200 mL) was added and extracted with ethyl acetate (3×60 mL). The organic layers were combined, washed with brine (2×20 mL), dried over Na₂SO₄, filtered, and concentrated under reduced pressure to give a residue. The residue was co-evaporated with DCM (60 mL) three times to give MYCi 606 (5.2 g, 84.70% yield) as a white solid. 1H NMR (400 MHz, DMSO-d6) δ 3.77 (s, 3H), 4.78 (br s, 2H), 6.82-6.91 (m, 2H), 7.24-7.32 (m, 3H), 7.40 (d, J=8.00 Hz, 1H), 7.64-7.70 (m, 1H), 7.72-7.76 (m, 1H), 7.78 (s, 1H), 9.24 (s, 1H) LCMS (ESI+): m/z 596.9 (M+H)+, RT: 3.260 min 30_90AB_6 min-220-254-ELSD: 30-90AB_6 min-220-254-ELSD LC/MS The gradient was 30% B in 0.40 min and 30-90% B in 2.60 min, hold on 90% B in 1.00 min, and then 90-30% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Kinetex C18 2.1*50 mm, 5 um. Detection methods are diode array (DAD) and evaporative light scattering detection (ELSD). MS mode was positive electrospray ionization. MS range was 100-1000.

### Example 3: Biological Data of Selected Compounds

Biological data of MYCi 606 was determined relative to, among other compounds, one or more of MCYi 975, MYCi 605, and MYCi 7037.

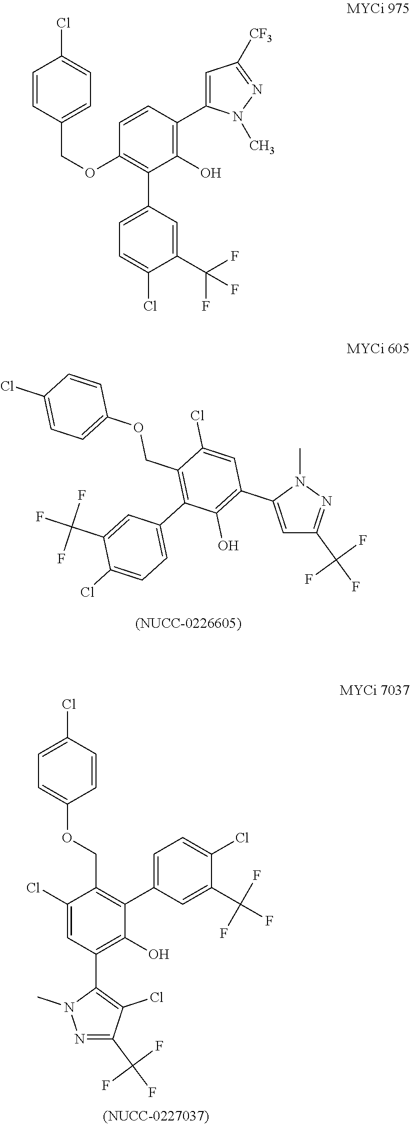 [SMILES: Cn1nc(C(F)(F)F)cc1-c1ccc(OCc2ccc(Cl)cc2)c(-c2ccc(Cl)c(C(F)(F)F)c2)c1O ; Cn1nc(C(F)(F)F)cc1-c1cc(Cl)c(COc2ccc(Cl)cc2)c(-c2ccc(Cl)c(C(F)(F)F)c2)c1O ; Cn1nc(C(F)(F)F)c(Cl)c1-c1cc(Cl)c(COc2ccc(Cl)cc2)c(-c2ccc(Cl)c(C(F)(F)F)c2)c1O]

Table 1 shows cell study data of MYCi 606 vs. MCYi 975, MYCi 605, and MYCi 7037. For IC50 determinations, PC3 and PC12 cells were treated with increasing concentrations of the indicated compounds for 72 hours, and cell viability was assessed using a CellTitreGlo Assay (Promega). For MYC and ATF4 protein level determination, PC3 cells were treated with the indicated compounds for 24-hours, and cell lysates were processed for western blot with MYC and ATF4 antibodies using Actin as a control. Quantification analyses were performed by Biorad ChemiDoc Imager and Bio-Rad Image Lab software.

TABLE 1

| | | | | Relative ATF4 |
|---|---|---|---|---|
| | | | | protein at |
| | | | Relative MYC | 10 μM (fold |
| | | | protein at 10 μM | change, PC3 |
| Molecule Name | PC3 IC50 (μM) | PC12 IC50 (μM) | (% PC3 24 h) | 24 h) |
| MYCi 7037 | 0.96 | 11.06 | 33.59 | 15.07 |
| MYCi 605 | 1 | 10.46 | 34.7 ± 3.2 | 10.3 ± 1.5 |
| MYCi 606 | 2.23 | 16.65 | 38.2 ± 3.9 | 9.7 ± 1.2 |
| MYCi 975 | 2.9 ± 0.5 | >16 | | |

Table 2 shows mouse study data of MYCi 606 vs. MYCi 975, MYCi 605, and MYCi 7037. Male FVB/N mice were inoculated with 1 million MycCaP:Pten-KO cells subcutaneously in their flanks. Once tumors were established (~200 mm³), mice were treated with the indicated compounds at 100 mg/kg/d p.o., and tumor volume was measured using calipers. Tumor growth inhibition (TGI) was determined after 14 days. Toxicity was assessed as −: 0-5% weight loss; +: 5-15% weight loss; ++: 15-20% weight loss; +++: >20% weight loss or death. Plasma levels of compounds (PK) were determined after 1 mg/kg dosing p.o. in female BALB/C mice. 1, Tl/2~4-6 hr; 2, Tl/2 8-12 h; 3, Tl/2>20 hr.

TABLE 2

| | | Efficacy MycCaP; | |
|---|---|---|---|
| | PK Relative to MYCi | Pten-ko (TGI @ | Toxicity @ 100 |
| Molecule Name | 975 | 100 mg/kg/d po) | mg/kg/d |
| MYCi 975 | 1 | 50 | − |
| MYCi 605 | 2 | 90 | ++ |
| MYCi 606 | 3 | 75 | − |
| MYCi 7037 | 3 | 80 | ++ |

Table 3 shows plasma pharmacokinetic parameters of MYCi 606 vs. MYCi 975 and MYCi 605 after p.o. (1 mg/kg) administration to Female C57BL/6 mice. Plasma levels of the compounds were determined after 1 mg/kg dosing P0 in female BALB/C mice.

TABLE 3

| | T_{1/2}_range* | T_{1/2} | Tₘₐₓ | Cₘₐₓ | AUC₀₋ₜ |
|---|---|---|---|---|---|
| Compound | (h) | (h) | (h) | (ng/mL) | (ng · h/mL) |
| MYCi 975 | 8-24 | 4.16 | 6 | 439 | 5646 |
| MYCi 605 | | | 8 | 274 | 4829 |
| MYCi 606 | | | 12 | 841 | 15194 |

| | AUC_{0-∞} | AUMC₀₋ₜ | AUMC_{0-∞} | MRT_{INF} |
|---|---|---|---|---|
| | (ng · h/mL) | (h*h*ng/mL) | (h*h*ng/mL) | (h) |
| MYCi 975 | 5834 | 48358 | 53976 | 9.25 |
| MYCi 605 | | 49995 | | |
| MYCi 606 | | 177148 | | |

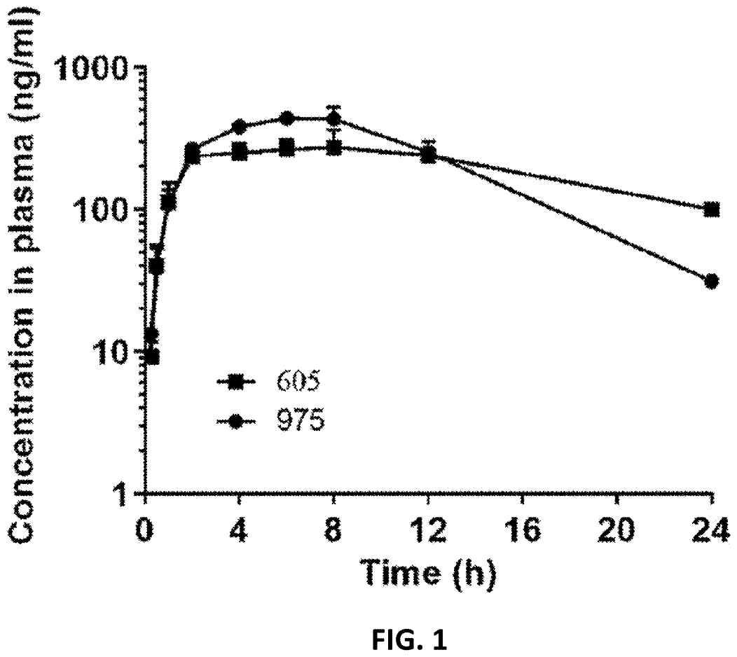

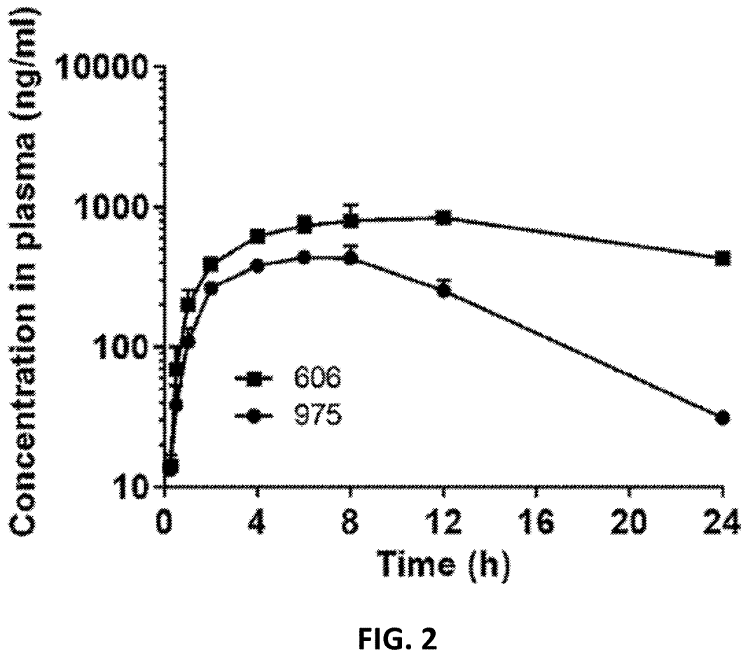

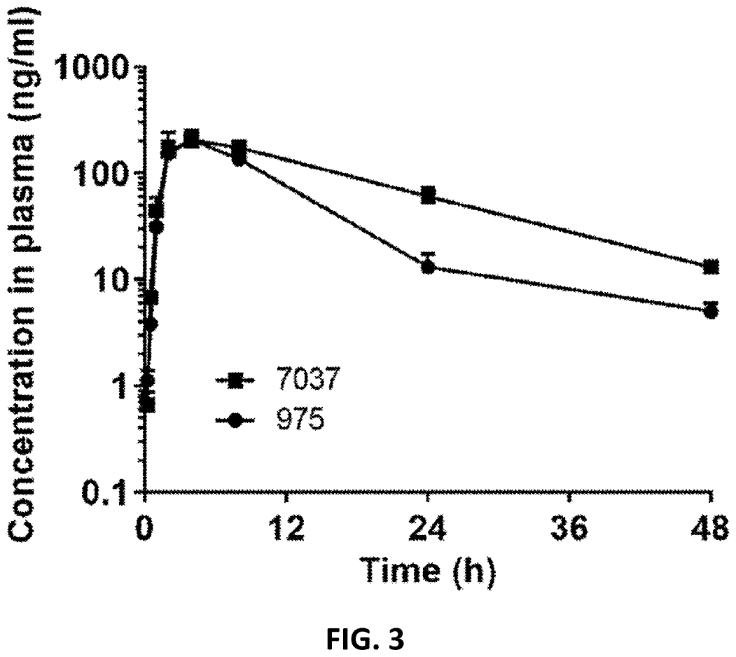

FIG. 1 shows the plasma levels of the compounds (ng/mL) overtime graphically for MYCi 605 relative to MYCi 975; FIG. 2 shows the plasma levels of the compounds (ng/mL) overtime graphically for MYCi 606 relative to MYCi 975; and FIG. 3 shows the plasma levels of the compounds (ng/mL) overtime graphically for MYCi 7037 relative to MYCi 975.

Table 4 shows tumor growth inhibition data for MYCi 606 vs. MCYi 975, MYCi 605, and MYCi 7037. Male FVB/N mice were inoculated with 1 million MycCaP; Pten-KO cells subcutaneously in their flanks. Once tumors are established (~200 mm³), mice were treated with the indicated compounds at 100 mg/kg/d, 30 mg/kg/d, and/or 10 mg/kg/d p.o, and tumor volume was measured using calipers. TGI was determined after 14 days.

TABLE 4

| Compound | Dosage | TGI |
|---|---|---|
| MYCi 975 | 100 mg/kg p.o., QD | 54.01% |
| MYCi 605 | 100 mg/kg p.o., QD | 92.51% |
| | 30 mg/kg p.o., QD | 65.67% |
| | 10 mg/kg p.o., QD | 56.29% |
| MYCi 606 | 100 mg/kg p.o., QD | 74.6% |
| | 30 mg/kg p.o., QD | 56.4% |
| | 10 mg/kg p.o., QD | 43.1% |
| MYCi 7037 | 100 mg/kg p.o., QD | 81.48% |
| | 30 mg/kg p.o., QD | 57.24% |
| | 10 mg/kg p.o., QD | 43.99% |

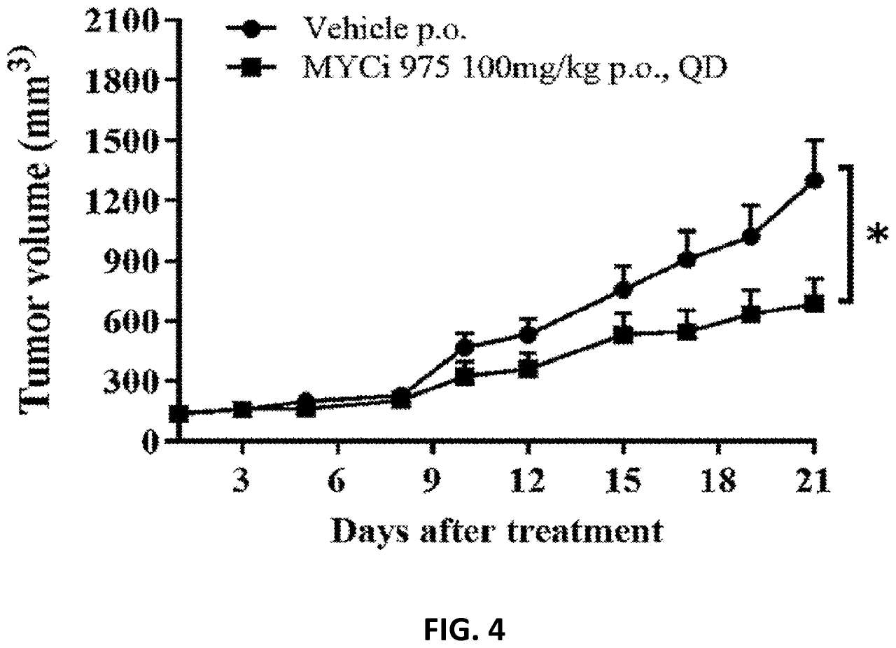

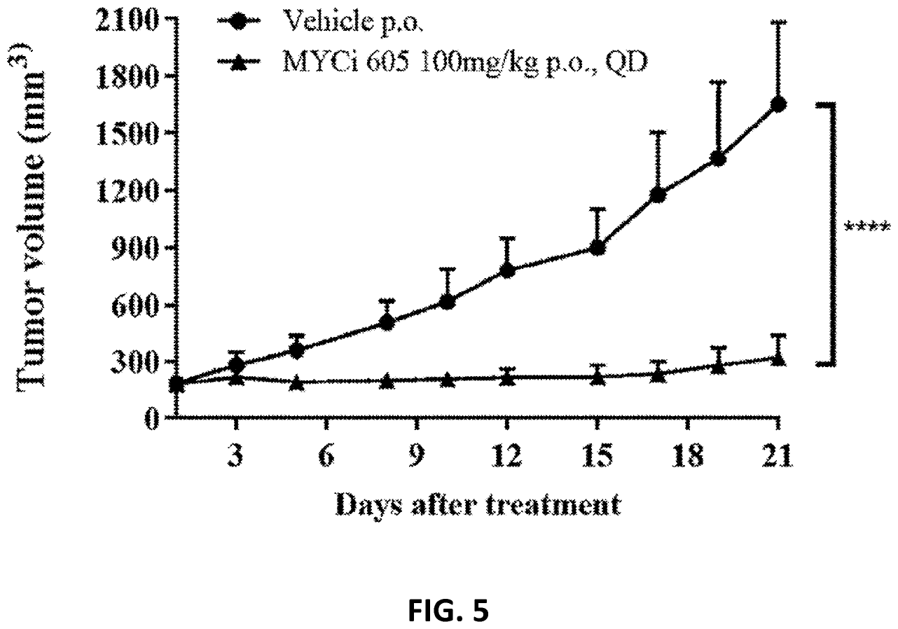

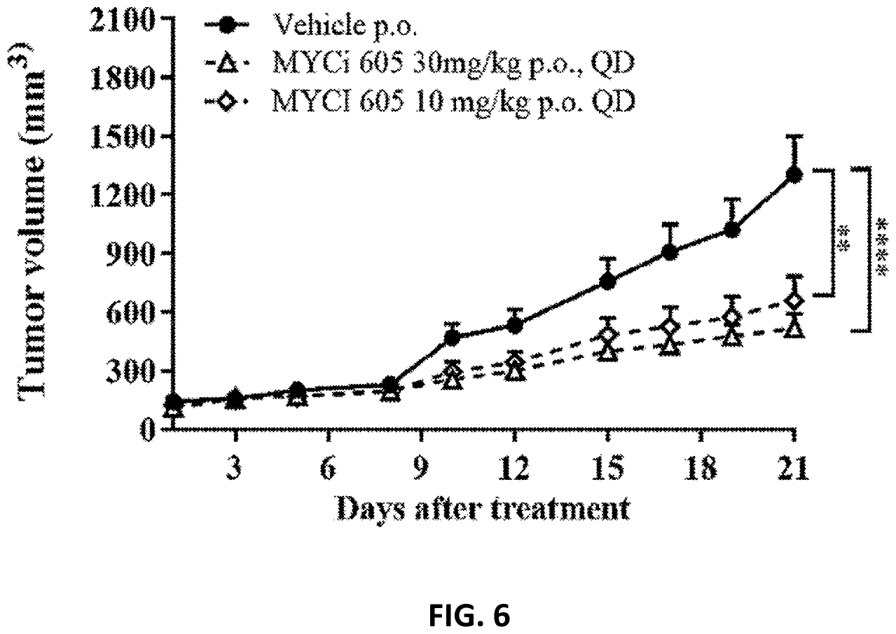

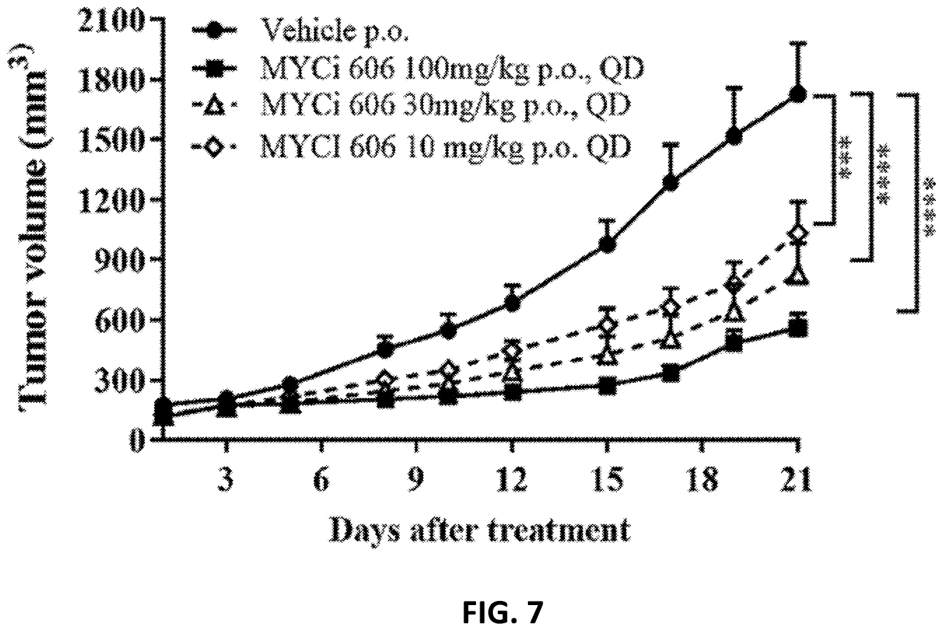

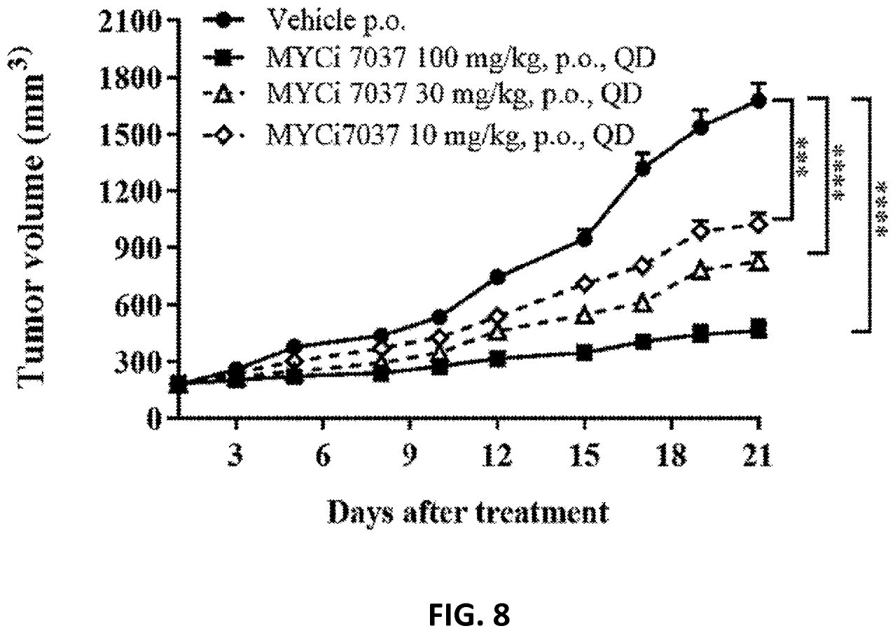

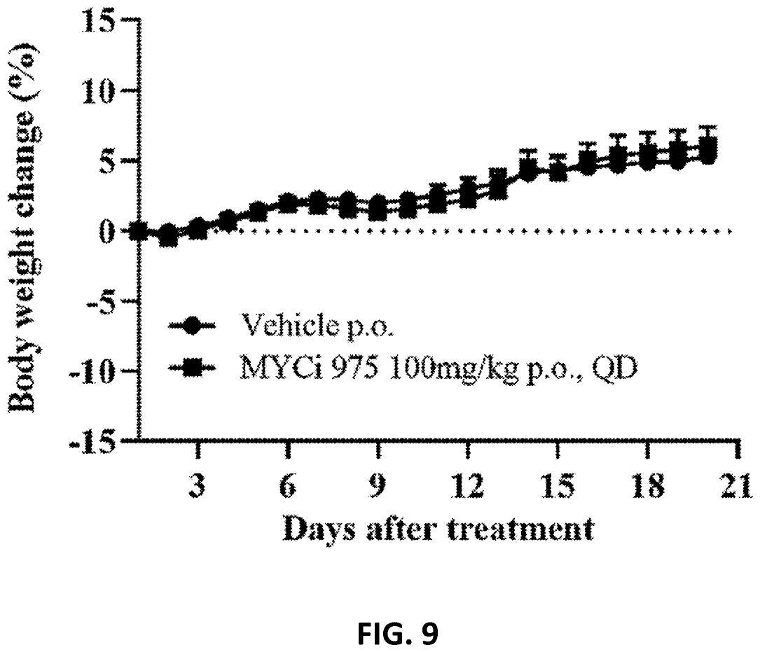

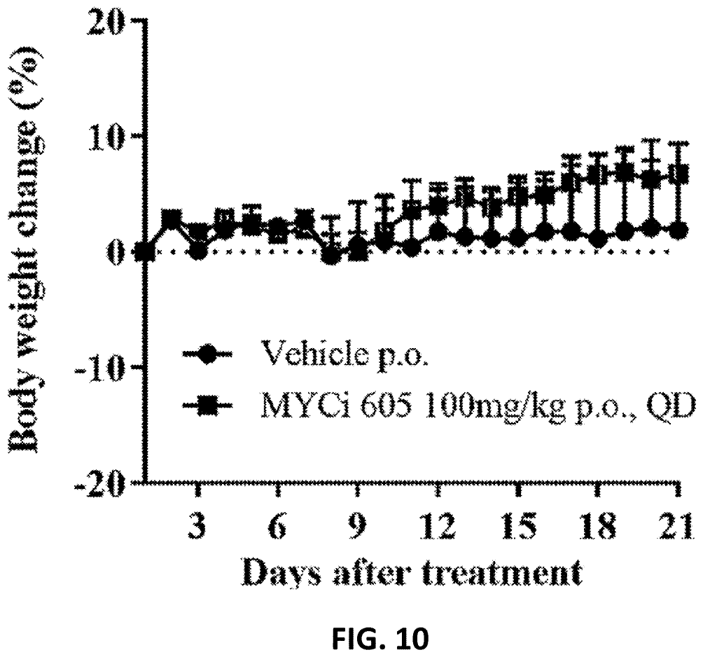

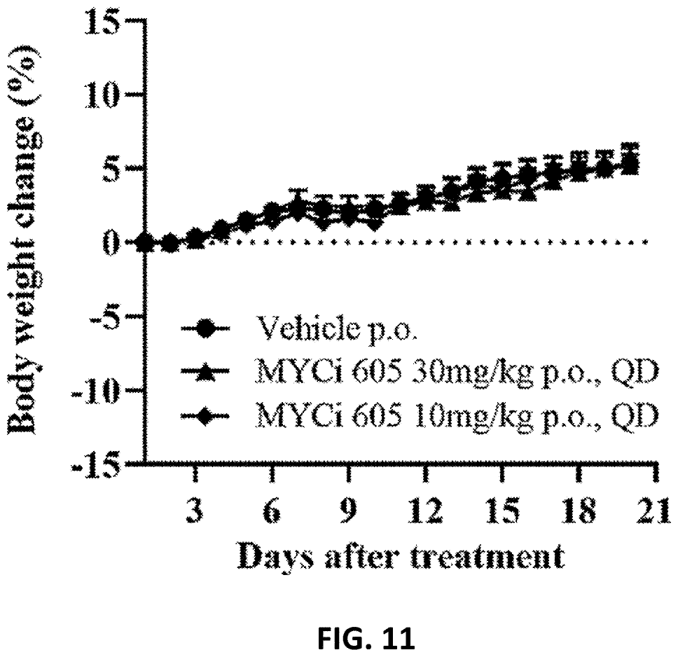

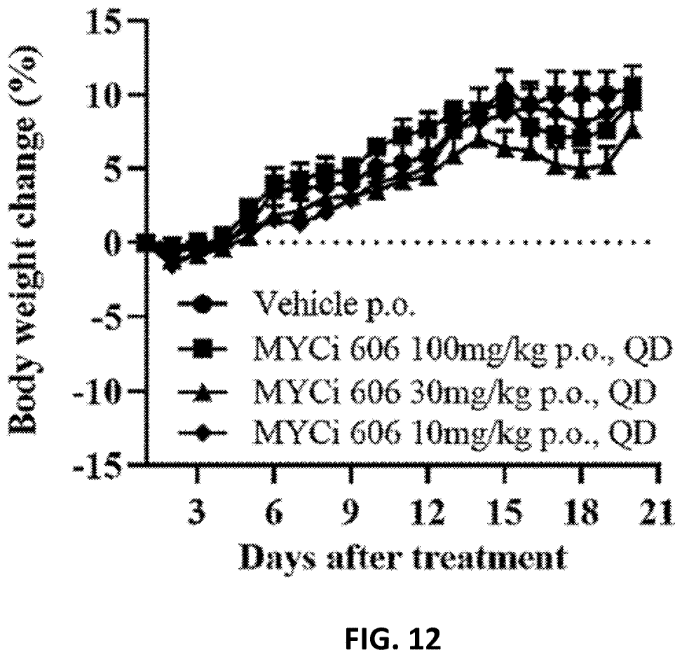

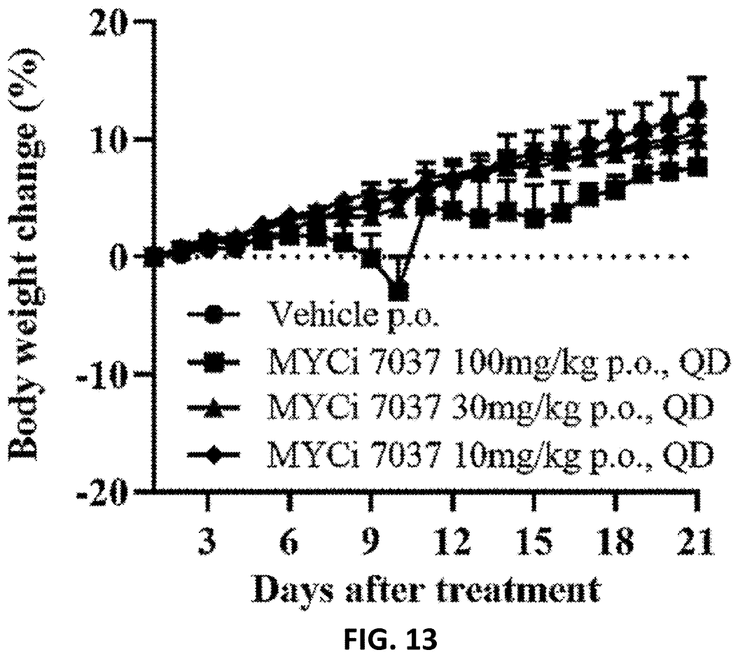

FIG. 4 depicts the tumor volume change in tested mice after oral administration of 100 mg/kg of MYCi 975 to the mice once per day. FIG. 5 depicts the tumor volume change in tested mice after oral administration of 100 mg/kg of MYCi 605 to the mice once per day. FIG. 6 depicts the tumor volume change in tested mice after oral administration of 30 mg/kg and 10 mg/kg of MYCi 605 to the mice once per day. FIG. 7 depicts the tumor volume change in tested mice after oral administration of 100 mg/kg, 30 mg/kg, and 10 mg/kg of MYCi 606 to the mice once per day. FIG. 8 depicts the tumor volume change in tested mice after oral administration of 100 mg/kg, 30 mg/kg, and 10 mg/kg of MYCi 7037 to the mice once per day. FIG. 9 depicts the body weight change in tested mice after oral administration of 100 mg/kg of MYCi 975 to the mice once per day. FIG. 10 depicts the body weight change in tested mice after oral administration of 100 mg/kg of MYCi 605 to the mice once per day. FIG. 11 depicts the body weight change in tested mice after oral administration of 30 mg/kg and 10 mg/kg of MYCi 605 to the mice once per day. FIG. 12 depicts the body weight change in tested mice after oral administration of 100 mg/kg, 30 mg/kg, and 10 mg/kg of MYCi 606 to the mice once per day. FIG. 13 depicts the body weight change in tested mice after oral administration of 100 mg/kg, 30 mg/kg, and 10 mg/kg of MYCi 7037 to the mice once per day.

1. A compound having Formula I:

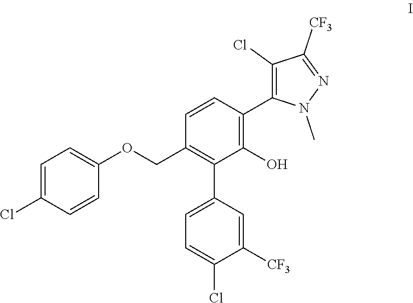 [SMILES: Cn1nc(C(F)(F)F)c(Cl)c1-c1ccc(COc2ccc(Cl)cc2)c(-c2ccc(Cl)c(C(F)(F)F)c2)c1O]

2. A pharmaceutical composition, the pharmaceutical composition comprising the compound of claim 1 and a pharmaceutical carrier, excipient, or diluent.

3. A method for treating cancer, the method comprising administering the compound of claim 1 or a pharmaceutical composition thereof to a subject having the cancer.

4. The method of claim 3, wherein the cancer is selected from multiple myeloma, leukemia, non-small cell lung cancer, colon cancer, cancer of the central nervous system, melanoma, ovarian cancer, renal cancer, prostate cancer, and breast cancer.
